(19) United States
(12) Patent Application Publication
(10) Pub. No.: US 2025/0087303 A1
(43) Pub. Date: Mar. 13, 2025

(54) Nucleic Acid Sequences Encoding Repeated Sequences Resistant to Recombination in Viruses

(71) Applicant: Altius Institute for Biomedical Sciences, Seattle, WA (US)

(72) Inventors: Jennifer M. Cherone, Seattle, WA (US); Alister PW Funnell, Seattle, WA (US); Eric David Haugen, Seattle, WA (US); John A. Stamatoyannopoulos, Seattle, WA (US)

(21) Appl. No.: 18/720,443

(22) PCT Filed: Dec. 15, 2022
(86) PCT No.: PCT/US2022/081701

§ 371 Date: Jun. 14, 2024

Related U.S. Application Data
(60) Provisional application No. 63/291,137, filed on Dec. 17, 2021.

(51) Int. Cl. G16B 20/50; G16B 30/20

(52) U.S. Cl. CPC G16B 20/50; G16B 30/20

(57) ABSTRACT

The present disclosure provides a computer-implemented method for diversifying an initial nucleic acid sequence encoding a protein with repeating amino acid sequences. The initial nucleic acid sequence comprises a plurality of contiguous stretches of nucleotides that are identical. The method may involve identifying in the nucleic acid sequence, a first contiguous stretch of nucleotides (nts) that is identical in sequence to a second contiguous stretch of nts and is longer than 14 nts; and replacing, in the 2^{nd} contiguous stretch of nts, a codon encoding an amino acid with another codon encoding the same amino acid. The identifying and replacing is performed over the length of the nucleic acid sequence until there are no two contiguous stretches of nts that are identical in sequence and are longer than 14 nts.

### CROSS-REFERENCE

This application claims the benefit of U.S. Provisional Patent Application No. 63/291,137, filed Dec. 17, 2021, which application is incorporated herein by reference in its entirety.

### INCORPORATION-BY-REFERENCE OF MATERIAL ELECTRONICALLY SUBMITTED

A Sequence Listing is provided herewith as a Sequence Listing XML, “ALTI-733WO_SEQ_LIST” created on Dec. 15, 2022, and having a size of 61,000 bytes. The contents of the Sequence Listing XML are incorporated by reference herein in their entirety.

### INTRODUCTION

Viruses, e.g., lentiviruses tend to recombine nucleic acid sequences that include repeated sequences. Nucleic acid sequences that encode polypeptides with repeated amino acid sequences often include contiguous stretches of nucleotides that repeat throughout the nucleic acid sequences. Lentiviruses are preferred for human gene therapy. However, lentiviruses frequently delete repeated stretches of nucleotides resulting in reduced production of viruses carrying the entire therapeutic gene. While others have attempted to overcome this problem by diversifying the amino acid sequence of the polypeptide, the resulting polypeptide usually has reduced activity.

Thus, there is a need for methods for increasing nucleic acid sequence diversity of nucleic acids encoding proteins with repeated amino acid sequences such that while the nucleic acid sequence is diversified the amino acid sequence of the encoded protein is unchanged.

### SUMMARY

The present disclosure provides a computer-implemented method for diversifying an initial nucleic acid sequence encoding a protein with repeating amino acid sequences. The initial nucleic acid sequence comprises a plurality of identical contiguous stretches of nucleotides (nts). The method may involve identifying in the nucleic acid sequence, a first contiguous stretch of nucleotides (nts) that is identical in sequence to a second contiguous stretch of nts and is longer than 14 nts; and replacing, in the 2^{nd} contiguous stretch of nts, a codon encoding an amino acid with another codon encoding the same amino acid. The identifying and replacing is performed over the length of the nucleic acid sequence until there are no two contiguous stretches of nts that are identical in sequence and are longer than 14 nts. The method may be performed on a nucleic acid sequence encoding a DNA binding domain (DBD) comprising transcription activator-like effector (TALE) repeat units (RUs).

### BRIEF DESCRIPTION OF THE DRAWINGS

FIGS. 1A-1E depict examples of computer-implemented methods for performing diversification of an initial nucleic acid sequence comprising multiple contiguous stretches of nucleotides that are identical.

FIG. 2 shows increased amount of a lentiviral vector that contains a diversified nucleic acid sequence (designated as “DR A11” in panel A and as “DR” in panels B and C) as compared to the amount of a lentiviral vector that contains a nucleic acid sequence that includes multiple contiguous stretches of nucleotides that are identical (designated as “WT A11” in panel A and as “A11” or “WT” in panel C).

### DETAILED DESCRIPTION

Before exemplary aspects of the present invention are described, it is to be understood that this invention is not limited to particular aspects described, as such may, of course, vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and exemplary methods and materials may now be described. Any and all publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a protein” includes a plurality of such proteins and reference to “the polynucleotide” includes reference to one or more polynucleotides, and so forth.

It is further noted that the claims may be drafted to exclude any element which may be optional. As such, this statement is intended to serve as antecedent basis for use of such exclusive terminology as “solely”, “only” and the like in connection with the recitation of claim elements, or the use of a “negative” limitation.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed. To the extent such publications may set out definitions of a term that conflicts with the explicit or implicit definition of the present disclosure, the definition of the present disclosure controls.

As will be apparent to those of skill in the art upon reading this disclosure, each of the individual aspects described and illustrated herein has discrete components and features which may be readily separated from or combined with the features of any of the other several aspects without departing from the scope or spirit of the present invention. Any recited method can be carried out in the order of events recited or in any other order which is logically possible.

### Definitions

As used herein, the term “plurality” refers to two or more.

As used herein, the term “derived” in the context of a polypeptide refers to a polypeptide that has a sequence that is based on that of a protein from a particular source (e.g., an animal pathogen such as Legionella or a plant pathogen such as Xanthomonas). A polypeptide derived from a protein from a particular source may be a variant of the protein from the particular source (e.g., an animal pathogen such as Legionella or a plant pathogen such as Xanthomonas). For example, a polypeptide derived from a protein from a particular source may have a sequence that is modified with respect to the protein's sequence from which it is derived. A polypeptide derived from a protein from a particular source shares at least 30% sequence identity with, at least 40% sequence identity with, at least 50% sequence identity with, at least 60% sequence identity with, at least 70% sequence identity with, at least 80% sequence identity with, or at least 90% sequence identity with the protein from which it is derived.

The DNA binding protein (DBP) disclosed herein may be derived from a nucleic acid binding domain of a DNA binding protein of an animal or plant pathogen. The term “modular” as used herein in the context of a nucleic acid binding domain, e.g., a modular DNA binding polypeptide (e.g., a Xanthomonas TALE or a modular animal pathogen-DNA binding polypeptide “MAP-DBP”) indicates that the plurality of repeat units present in the DBP can be rearranged and/or replaced with other repeat units and can be arranged in an order such that the DBP binds to the target nucleic acid. For example, any repeat unit in a modular nucleic acid binding domain can be switched with a different repeat unit. In some aspects, modularity of the nucleic acid binding domains disclosed herein allows for switching the target nucleic acid base for a particular repeat unit by simply switching it out for another repeat unit. In some aspects, modularity of the nucleic acid binding domains disclosed herein allows for swapping out a particular repeat unit for another repeat unit to increase the affinity of the repeat unit for a particular target nucleic acid. Overall, the modular nature of the nucleic acid binding domains disclosed herein enables the development of DBP that can precisely target any nucleic acid sequence of interest.

The terms “polypeptide,” “peptide,” and “protein”, used interchangeably herein, refer to a polymeric form of amino acids of any length, which can include genetically coded and non-genetically coded amino acids, chemically or biochemically modified or derivatized amino acids, and polypeptides having modified polypeptide backbones. The terms include fusion proteins, including, but not limited to, fusion proteins with a heterologous amino acid sequence, fusion proteins with heterologous and homologous leader sequences, with or without N-terminus methionine residues; immunologically tagged proteins; and the like. In specific aspects, the terms refer to a polymeric form of amino acids of any length which include genetically coded amino acids. In particular aspects, the terms refer to a polymeric form of amino acids of any length which include genetically coded amino acids fused to a heterologous amino acid sequence.

The term “heterologous” refers to two components that are defined by structures derived from different sources. For example, in the context of a polypeptide, a “heterologous” polypeptide may include operably linked amino acid sequences that are derived from different polypeptides (e.g., a DBD and a functional domain derived from different sources). Similarly, in the context of a polynucleotide encoding a chimeric polypeptide, a “heterologous” polynucleotide may include operably linked nucleic acid sequences that can be derived from different genes or nucleic acids sequences that encode a polypeptide that does not exist in nature or a polypeptide comprising a first sequence and a second sequence from two or more different polypeptides. Other exemplary “heterologous” nucleic acids include expression constructs in which a nucleic acid comprising a coding sequence is operably linked to a regulatory element (e.g., a promoter) that is from a genetic origin different from that of the coding sequence (e.g., to provide for expression in a host cell of interest, which may be of different genetic origin than the promoter, the coding sequence or both). In the context of recombinant cells, “heterologous” can refer to the presence of a nucleic acid (or gene product, such as a polypeptide) that is of a different genetic origin than the host cell in which it is present.

The term “operably linked” refers to linkage between molecules to provide a desired function. For example, “operably linked” in the context of nucleic acids refers to a functional linkage between nucleic acid sequences. By way of example, a nucleic acid expression control sequence (such as a promoter, signal sequence, or array of transcription factor binding sites) may be operably linked to a second polynucleotide, wherein the expression control sequence affects transcription and/or translation of the second polynucleotide. In the context of a polypeptide, “operably linked” refers to a functional linkage between amino acid sequences (e.g., different domains) to provide for a described activity of the polypeptide.

As used herein, the term “cleavage” refers to the breakage of the covalent backbone of a nucleic acid, e.g., a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain aspects, the polypeptides provided herein are used for targeted double-stranded DNA cleavage.

A “cleavage half-domain” is a polypeptide sequence which, in conjunction with a second polypeptide (either identical or different) forms a complex having cleavage activity (preferably double-strand cleavage activity).

A “target nucleic acid,” “target sequence,” or “target site” is a nucleic acid sequence that defines a portion of a nucleic acid to which a binding molecule, such as, the DBP disclosed herein will bind. The target nucleic acid may be present in inside a cell. A target nucleic acid may be present in a regulatory region, e.g., promoter sequence, of a target gene whose expression is to be modulated by the DBP.

An “exogenous” molecule is a molecule that is not normally present in a cell but can be introduced into a cell by one or more genetic, biochemical or other methods. An exogenous molecule can comprise, for example, a functioning version of a malfunctioning endogenous molecule, e.g., a gene or a gene segment lacking a mutation present in the endogenous gene. An exogenous nucleic acid can be present in an infecting viral genome, a plasmid or episome introduced into a cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “gene,” for the purposes of the present disclosure, includes a DNA region encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control region.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA, shRNA, RNAi, miRNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristylation, and glycosylation.

“Modulation” of gene expression refers to a change in the activity of a gene. Modulation of expression can include, but is not limited to, gene activation and gene repression. Genome editing (e.g., cleavage, alteration, inactivation, donor integration, random mutation) can be used to modulate expression. Gene inactivation refers to any reduction in gene expression as compared to a cell that does not include a polypeptide or has not been modified by a polypeptide as described herein. Thus, gene inactivation may be partial or complete.

The terms “patient” or “subject” are used interchangeably to refer to a human or a non-human animal (e.g., a mammal).

The terms “treat”, “treating”, treatment” and the like refer to a course of action (such as administering a polypeptide or a nucleic acid encoding the polypeptide or a cell comprising the nucleic acid encoding the polypeptide or expressing the polypeptide) initiated after a disease, disorder or condition, or a symptom thereof, has been diagnosed, observed, and the like so as to eliminate, reduce, suppress, mitigate, or ameliorate, either temporarily or permanently, at least one of the underlying causes of a disease, disorder, or condition afflicting a subject, or at least one of the symptoms associated with a disease, disorder, condition afflicting a subject.

The terms “prevent”, “preventing”, “prevention” and the like refer to a course of action (such as administering a polypeptide or a nucleic acid encoding the polypeptide or a cell comprising the nucleic acid encoding the polypeptide or expressing the polypeptide) initiated in a manner (e.g., prior to the onset of a disease, disorder, condition or symptom thereof) so as to prevent, suppress, inhibit or reduce, either temporarily or permanently, a subject's risk of developing a disease, disorder, condition or the like (as determined by, for example, the absence of clinical symptoms) or delaying the onset thereof, generally in the context of a subject predisposed to having a particular disease, disorder or condition. In certain instances, the terms also refer to slowing the progression of the disease, disorder or condition or inhibiting progression thereof to a harmful or otherwise undesired state.

The phrase “therapeutically effective amount” refers to the administration of an agent to a subject, either alone or as a part of a pharmaceutical composition and either in a single dose or as part of a series of doses, in an amount that is capable of having any detectable, positive effect on any symptom, aspect, or characteristics of a disease, disorder or condition when administered to a patient. The therapeutically effective amount can be ascertained by measuring relevant physiological effects.

The terms “conjugating,” “conjugated,” and “conjugation” refer to an association of two entities, for example, of two molecules such as two proteins, two domains (e.g., a binding domain and a cleavage domain), or a protein and an agent, e.g., a protein binding domain and a small molecule. The association can be, for example, via a direct or indirect (e.g., via a linker) covalent linkage or via non-covalent interactions. In some aspects, the association is covalent. In some aspects, two molecules are conjugated via a linker connecting both molecules. For example, in some aspects where two proteins are conjugated to each other, e.g., a binding domain and a cleavage domain of an engineered nuclease, to form a protein fusion, the two proteins may be conjugated via a polypeptide linker, e.g., an amino acid sequence connecting the C-terminus of one protein to the N-terminus of the other protein. Such conjugated proteins may be expressed as a fusion protein.

The term “consensus sequence,” as used herein in the context of nucleic acid or amino acid sequences, refers to a sequence representing the most frequent nucleotide/amino acid residues found at each position in a plurality of similar sequences. Typically, a consensus sequence is determined by sequence alignment in which similar sequences are compared to each other. A consensus sequence of a protein can provide guidance as to which residues can be substituted without significantly affecting the function of the protein.

As used herein, the term “genome modifying proteins” refer to nucleic acid binding domains and functional domains which cooperate to modify genome or epigenome is a cell. Examples of genome modifying proteins are provided herein and include but are not limited to nucleic acid binding proteins comprising modular repeat units, nucleic acid binding proteins comprising zinc fingers, functional domains such as labels, tags, polypeptides having nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity, e.g., nucleases, transcriptional activators, transcriptional repressors, chromatin modifying protein, and the like. Genome modifying proteins also encompass a single polypeptide comprising a nucleic acid binding domain and functional domain or two or more polypeptides, where a first polypeptide comprises a nucleic acid binding domain and a second polypeptide comprises a functional domain and wherein the first and second polypeptide associate with each other via a non-covalent interaction, such as, via a interactions mediated by first and second members of a heterodimer, where one of the first and second polypeptide is conjugated to the first member and the other polypeptide is conjugated to the second member.

As used herein, a “fusion protein” includes a first protein moiety, e.g., a nucleic acid binding domain, having a peptide linkage with a second protein moiety. In certain aspects, the fusion protein is encoded by a single fusion gene.

“Domain” is used to describe a segment of a protein or nucleic acid. Unless otherwise indicated, a domain is not required to have any specific functional property.

As used herein, the term “gene therapy” refers to the introduction of extra genetic material into the total genetic material in a cell that restores, corrects, or modifies expression of a gene or gene product, or for the purpose of expressing a therapeutic polypeptide. In particular aspects, introduction of genetic material into the cell's genome for the purpose of expressing a therapeutic polypeptide is considered gene therapy.

### Methods

The present disclosure provides a method for diversifying a nucleic acid sequence encoding a protein with repeating amino acid sequences. The method can be implemented using a computer. The nucleic acid sequence comprises a plurality of contiguous stretches of identical sequences. The method may involve (a) identifying in the nucleic acid sequence, a 1^{st} contiguous stretch of nucleotides (nts) that is identical in sequence to a 2^{nd} contiguous stretch of nts and is longer than 14 nts; (b) replacing, in the 2^{nd} contiguous stretch of nts, a codon encoding an amino acid with another codon encoding the same amino acid; (c) determining whether the replaced codon introduces a restriction enzyme (RE) site, (i) retaining the replaced codon if a RE site is not introduced or (ii) if a RE site is introduced, (1) reverting to the original codon or (2) replacing the codon with another codon encoding the same amino acid; and (d) repeating steps (a)-(c) until a diversified nucleic acid sequence is generated which diversified nucleic acid sequence does not include a pair of identical contiguous stretches of nts that are longer than 14 nts. If step (c) (ii) (2) is performed, then step (c) may be repeated to determine whether the replaced codon introduces a RE site. In certain embodiments, steps (a)-(d) may be conducted simultaneously on an initial nucleic acid sequence encoding a protein with repeating amino acid sequences.

In certain embodiments, step (d) may be performed at least 50 times, at least 100 times, or at least 500 times. In certain embodiments, step (d) may be performed up to 2000 times or up to 1000 times. For example, step (d) may be performed 50-100 times, 100-200 times, 200-300 times, 300-400 times, 400-500 times, 500-600 times, 600-700 times, 700-800 times, 800-900 times, 900-10,000 times, e.g., 50-2000 times, 100-1000 times, 100-2000 times, 500-1000 times, or 500-2000 times.

In certain embodiments, steps a)-d) may be performed at least 5 times, at least 10 times, at least 100 times, or at least 500 times on the initial nucleic acid sequence. In certain embodiments, steps a)-d) may be performed up to 100 times or up to 200 times on the initial nucleic acid sequence. For examples, steps a)-d) may be performed 5-100 times, 5-200 times, 10-200 times, 10-200 times, 50-200 times, or 50-200 times.

In certain embodiments, after step (c), a first nucleic acid sequence may be generated or a plurality of first nucleic acid sequences may be generated that differ in sequence from the initial nucleic acid sequence. Step (d) is then performed on first nucleic acid sequence or the plurality of first nucleic acid sequences. In other words, step (d) is performed on the sequence generated from replacing a codon rather than on the initial nucleic acid sequence.

In certain embodiments, after step (c), the initial nucleic acid sequence is unchanged (e.g., due to creation of a RE and reverting to the original codon) and step (d) is performed on the initial nucleic acid sequence until a first nucleic acid sequence may be generated or a plurality of first nucleic acid sequences that differ in sequence from the initial nucleic acid sequence. Step (d) is then performed on the first nucleic acid sequence or the plurality of first nucleic acid sequences which may then generate a second nucleic acid sequence or a plurality of second nucleic acid sequences having a sequence different from the first nucleic acid sequence or the plurality of first nucleic acid sequences, respectively. Thus, the term “initial nucleic acid sequence” refers to the nucleic acid sequence that is diversified by steps (a)-(d). The terms “first nucleic acid sequence,” “second nucleic acid sequence,” and the so forth refer to the sequences generated after steps (a)-(d) which have a different sequence as compared to the initial nucleic acid sequence. The terms “1^{st} contiguous stretch of nucleotides” and “2^{nd} contiguous stretch of nucleotides” in step (a) refer to any pair of sequences that are identical over a length of 15 nts or longer. When step (a) is repeated it may be performed on the same pair or a different pair of sequences.

In certain embodiments, the method for generating a diversified nucleic acid sequence from the initial nucleic acid sequence may be performed in less than 5 hours, less than 1 hours, less than 30 minutes, less than 10 minutes, or less than 1 minute.

In certain embodiments, the method is performed until the length of a contiguous stretch of nts that is identical between a 1^{st} and a 2^{nd} nucleic acid segments is 13 nts or shorter, e.g., 12 nts, 11 nts, 10 nts, or shorter. In certain embodiments, the method is performed until the nucleic acid sequence contains no contiguous stretches of 14 nts that are identical to another contiguous stretch of 14 nts in the nucleic acid sequence. In certain embodiments, the method is performed until the nucleic acid sequence contains no contiguous stretches of 13 nts that are identical to another contiguous stretch of 13 nts in the nucleic acid sequence. In certain embodiments, the method is performed until the nucleic acid sequence contains no contiguous stretches of 12 nts that are identical to another contiguous stretch of 12 nts in the nucleic acid sequence. In certain embodiments, the method is performed until the nucleic acid sequence contains no contiguous stretches of 11 nts that are identical to another contiguous stretch of 11 nts in the nucleic acid sequence. In certain embodiments, the method is performed until the nucleic acid sequence contains no contiguous stretches of 10 nts that are identical to another contiguous stretch of 10 nts in the nucleic acid sequence.

In certain embodiments, the initial nucleic acid sequence, prior to the diversification, may include identical stretches of nucleotides that are each 15 nts long or longer, e.g., up to 200 nts, such as 15-200 nts, 18-200 nts, 20-200 nts, 25-200 nts, 30-200 nts, 35-200 nts, 40-200 nts, 45-200 nts, 50-200 nts, 55-200 nts, 15-100 nts, 18-100 nts, 20-100 nts, 25-100 nts, 30-100 nts, 35-100 nts, 40-100 nts, 45-100 nts, 50-100 nts, or 55-100 nts.

In certain embodiments, the method may generate a plurality of different diversified nucleic acid sequences from the initial nucleic acid sequence that all meet the criteria of comprising no continuous stretches of nts that are identical to another continuous stretch of nts where the identical contiguous stretches of nts are over 14 nts long. The method may then further involve ranking the plurality of different diversified nucleic acid sequences and selecting the highest ranked nucleic acid sequence(s). Ranking the plurality of different diversified nucleic acid sequences may involve one, two, or three different ranking methods as disclosed herein.

In certain embodiments, ranking the plurality of different diversified nucleic acid sequences may involve measuring lengths of identical contiguous stretches of nts within a diversified sequence and outputting the length of the longest pair of identical contiguous stretches of nts and repeating the measuring and outputting steps for each of the plurality of different diversified nucleic acid sequences. A diversified sequence that includes the shorter longest pair of identical contiguous stretches of nts is ranked higher than a diversified sequence that includes a longer longest pair of identical contiguous stretches of nts. For example, a 1^{st} diversified nucleic acid sequence may include a 1^{st} pair of identical contiguous stretch of nucleotides that are 13 nts long, a 2^{nd} pair of identical contiguous stretch of nucleotides that are 12 nts long, pair of identical contiguous stretch of nucleotides that are 11 nts long, a pair of identical contiguous stretch of nucleotides that are 10 nts long, and so on. The length of the longest pair of identical contiguous stretches of nts for this 1^{st} diversified nucleic acid sequence may be outputted as 13 nts. A 2^{nd} diversified nucleic acid sequence may include a 1^{st} pair of identical contiguous stretch of nucleotides that are 12 nts long, a 2^{nd} pair of identical contiguous stretch of nucleotides that are 11 nts long, pair of identical contiguous stretch of nucleotides that are 10 nts long, a pair of identical contiguous stretch of nucleotides that are 9 nts long, and so on. The length of the longest pair of identical contiguous stretches of nts for this 2^{nd} diversified nucleic acid sequence may be outputted as 12 nts. In this embodiment, since the 2^{nd} diversified sequence has the shorter longest pair of identical contiguous stretches of nts, it is ranked higher than the 1^{st} diversified nucleic acid sequence.

In certain embodiments, ranking the plurality of different diversified nucleic acid sequences may involve ranking the plurality of different diversified nucleic acid sequences based on percent divergence. In certain embodiments, only diversified nucleic acid sequences that have a minimum-percent-divergence of 16% may be retained while diversified nucleic acid sequences having a minimum-percent-divergence of less than 16% may be discarded. In certain embodiments, a minimum-percent-divergence may be calculated by comparing a plurality of pairs of 100 nts long stretches of nts within a diversified sequence and calculating a percent divergence for each pair. The lowest minimum-percent-divergence between any two 100 nts long stretches of nts within a diversified sequence is outputted. If the lowest minimum-percent-divergence is less than 16%, the diversified sequence may be discarded. The lowest minimum-percent-divergence may be calculated for each diversified sequence and used to rank the diversified sequences. For example, a 1^{st} diversified nucleic acid sequence may have a minimum-percent-divergence of 16%, a 2^{nd} diversified nucleic acid sequence may have a minimum-percent-divergence of 18%, a 3^{rd} diversified nucleic acid sequence may have a minimum-percent-divergence of 20%, and so on. In this embodiment, the 3^{rd} diversified nucleic acid sequence is ranked the highest while the 1^{st} diversified nucleic acid sequence is ranked the lowest.

In certain embodiments, ranking the plurality of different diversified nucleic acid sequences may involve identifying diversified nucleic acid sequences comprising a ratio of codons that is most similar to the ratio of codons in a cell in which the protein encoded by the diversified nucleic acid is to be expressed, e.g., mammalian cells, such as, human cell. Diversified nucleic acid sequences having a ratio of codons that is most similar to the ratio of codons in a host cell (i.e., cell in which the nucleic acid is to be expressed) is ranked the highest. In some embodiments, the ratio of codons most similar to the ratio of codons in a host cell may be a codon adaptation index (CAI). CAI may be calculated for each of the alternate diversified nucleic acid sequences and in some embodiments, only diversified nucleic acid sequences having a CAI of 0.7 or more may be selected for generation of the nucleic acid. CAI may be calculated as described in Sharp PM, Li WH. The codon Adaptation Index—a measure of directional synonymous codon usage bias, and its potential applications. Nucleic Acids Res. 1987; 15(3):1281-1295. doi:10.1093/nar/15.3.1281. CAI may also be calculated as described in Rice P., Longden I. and Bleasby A. EMBOSS: The European Molecular Biology Open Software Suite. Trends in Genetics. 2000 16(6):276-277.

In some embodiments, the plurality of alternate diversified nucleic acid sequences may first be ranked based on the length of the longest identical pair of sequences, with the diversified nucleic acid sequences having the shortest-longest identical pair of sequences being ranked the highest. In some embodiments, where there are multiple diversified nucleic acid sequences that each have the same shortest-longest of pairs of sequences, these diversified nucleic acid sequences may be ranked by minimum-percent-divergence, where diversified nucleic acid sequences that have the minimum-percent-divergence are ranked the highest. In some embodiments, where there are multiple diversified nucleic acid sequences that each have the same length of the longest identical pairs of sequences and same minimum-percent-divergence, these diversified nucleic acid sequences may be ranked by CAI, where the diversified nucleic acid sequences having a CAI closest to 1 are ranked higher than the diversified nucleic acid sequences having a CAI less than 1 and these diversified nucleic acid sequence(s) are selected for generating a nucleic acid. In embodiments, where the different diversified nucleic acid sequences have the same CAI, any of the diversified nucleic acid sequences may be selected for generating a nucleic acid.

In certain embodiments, the computer-implemented method disclosed herein may not involve codon optimization. Codon optimization is usually performed to increase expression level of the protein in the host cell. Codon optimization involves replacing a less frequently used codon in a nucleic acid sequence with a more frequently codon to increase expression of encoded protein in the host cell. In certain embodiments, the methods disclosed herein do not involve changing the amino acid sequence of the protein encoded by the initial nucleic acid sequence to achieve sequence diversification of the nucleic acid sequence. Rather, by replacing a codon with another codon encoding the same amino acid, the amino acid sequence of the encoded protein remains unchanged.

In certain embodiments, the method may be performed on nucleic acid sequences encoding DNA binding proteins comprising zinc finger proteins or transcription activator-like effector (TALE) repeat units. In certain embodiments, the DNA binding protein (DBP) may include a plurality of repeat units (RUs) arranged to bind to a target nucleic acid sequence. Each RU may comprise the sequence X₁₋₁₁X₁₂X₁₃X_{14-33, 34, or 35}. The DBP may also include a half-repeat unit as the last RU at the C-terminus of the protein. The half RU may comprise the amino acid sequence X₁₋₁₁X₁₂X₁₃X_{14-19, 20, or 21}. X₁₋₁₁ is a chain of 11 contiguous amino acids, X_{14-33 or 34 or 35} is a chain of 20, 21 or 22 contiguous amino acids, X_{14-20 or 21 or 22} is a chain of 7, 8 or 9 contiguous amino acids, X₁₂X₁₃ is a repeat variable diresidue (RVD) that is different in different RUs and determines which nucleotide of the target nucleic acid sequence the DBP binds to. The RVD can be selected from: (a) NH, HH, KH, NK, NQ, RH, RN, SS, NN, SN, KN, GN, VN, LN, DN, QN, EN, AN, or FN for binding to guanine (G); (b) NI, KI, RI, HI, CI, or SI for binding to adenine (A); (c) NG, HG, KG, RG, VG, IG, EG, MG, YG, AA, EP, VA, or QG for binding to thymine (T); (d) HD, RD, SD, ND, KD, AD, or YG for binding to cytosine (C); (e) NV or HN for binding to A or G; and (f) H*, HA, KA, N*, NA, NC, NS, RA, or S* for binding to A or T or G or C, wherein (*) means that the amino acid at X₁₃ is absent.

In certain embodiments, the nucleic acid sequence prior to the diversification as disclosed herein may include multiple contiguous stretches of nucleotides that each encode the amino acids X₁₋₁₁ of each of the RUs, where the amino acids X₁₋₁₁ are identical for each repeat unit and the half-repeat unit. Accordingly, the multiple contiguous stretches of nucleotides may also be identical. Similarly, the nucleic acid sequence may include multiple contiguous stretches of nucleotides that each encode the amino acids X_{14-33, 34, or 35} of each of the RUs, where the amino acids X_{14-33, 34, or 35} are identical for each repeat unit and 20, 21 or 22 contiguous amino acids of each repeat unit are identical to the 20, 21 or 22 contiguous amino acids of the half-RU. Accordingly, these multiple contiguous stretches of nucleotides may also be identical. In certain embodiments, the nucleic acid sequence may encode a DBP comprising 9.5 RUs, 11.5 RUs, 13.5 RUs, 16.5 RUs, 18.5RUs, 20.5 RUs, 21.5 RUs, or more, e.g., up to 30.5 RUs, 32.5 RUs, 35.5 RUs, 38.5 RUs, or 40.5 RUs. In addition, the RVDs may be identical in a plurality of the RUs resulting in contiguous stretches of nucleotides that encode RUs having identical sequences across the entire length of the RUs.

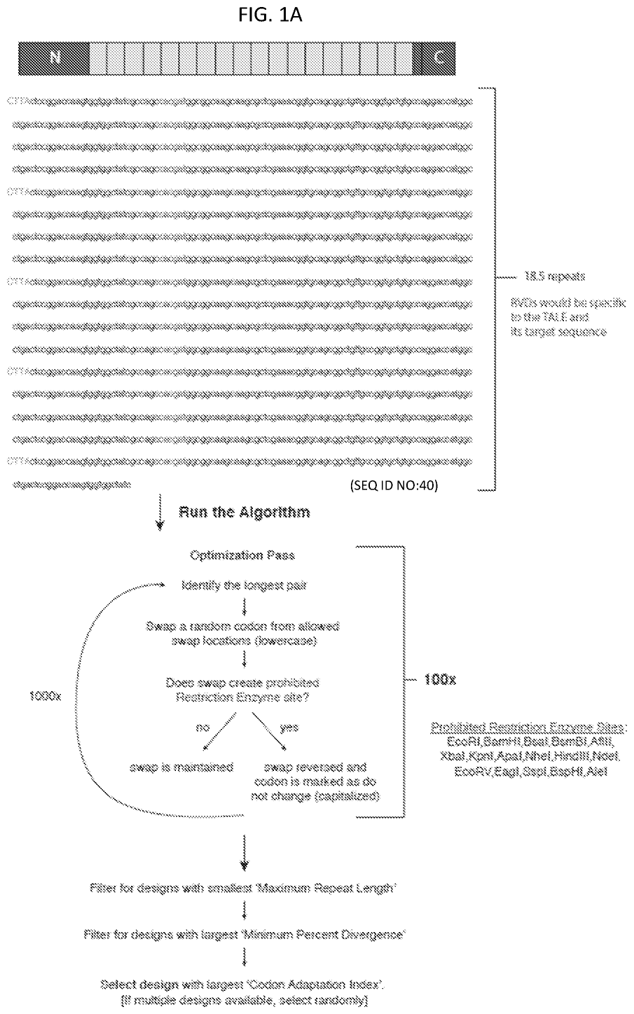

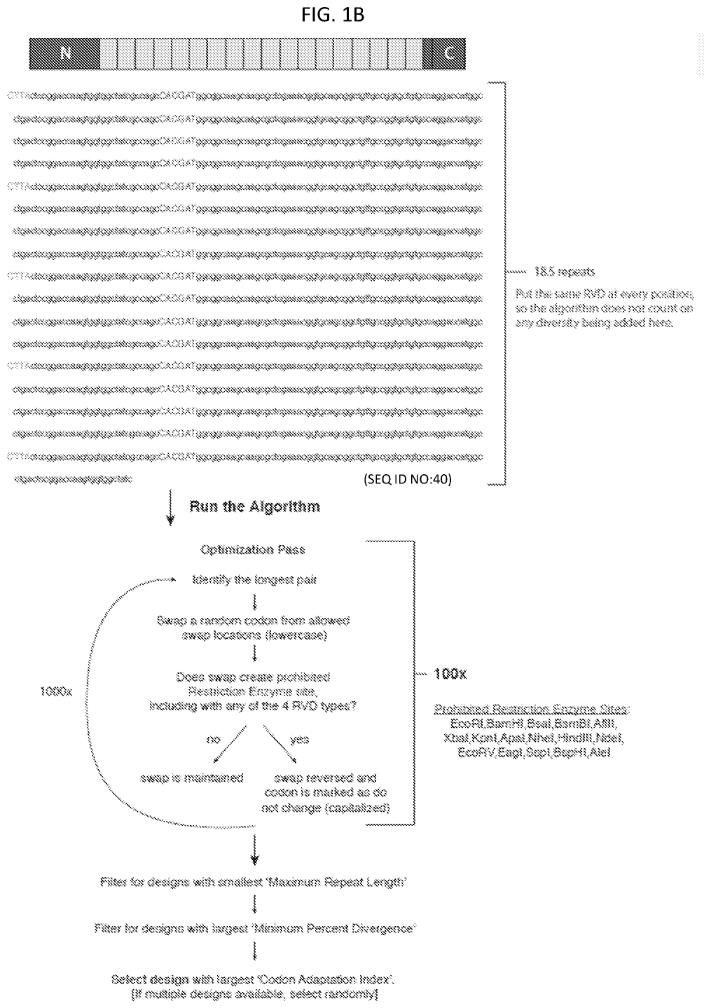

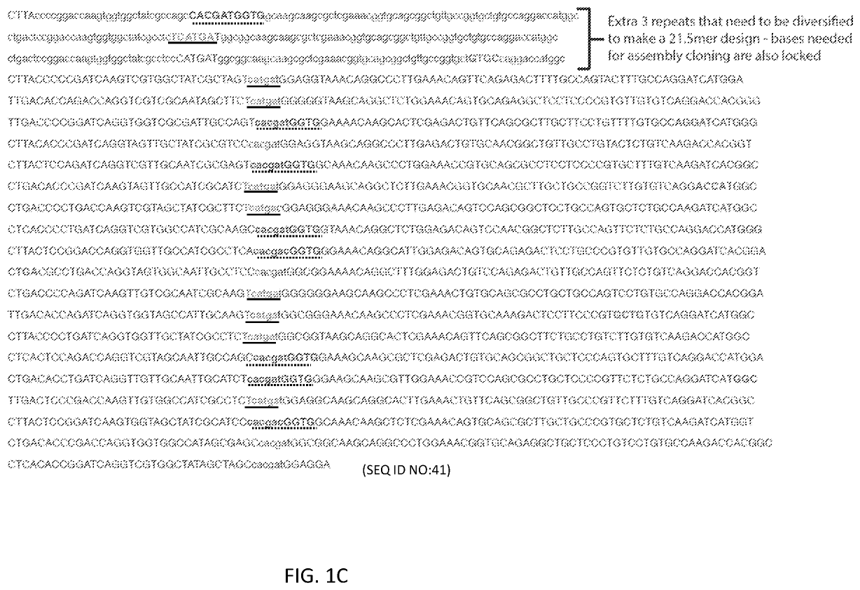

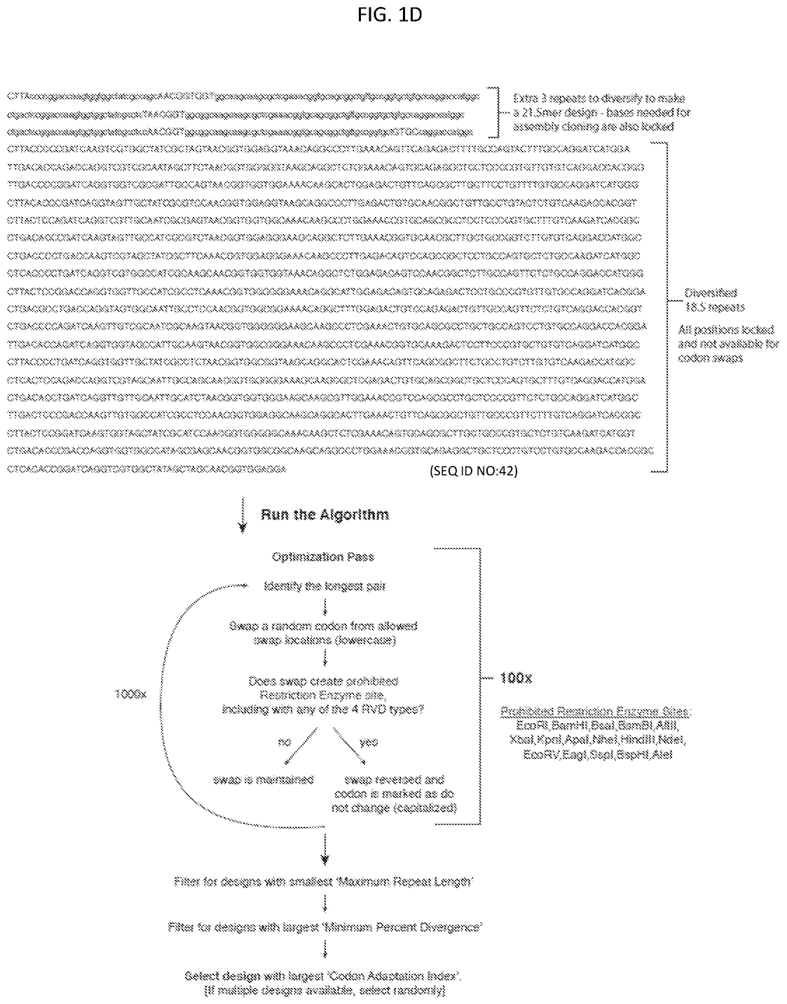

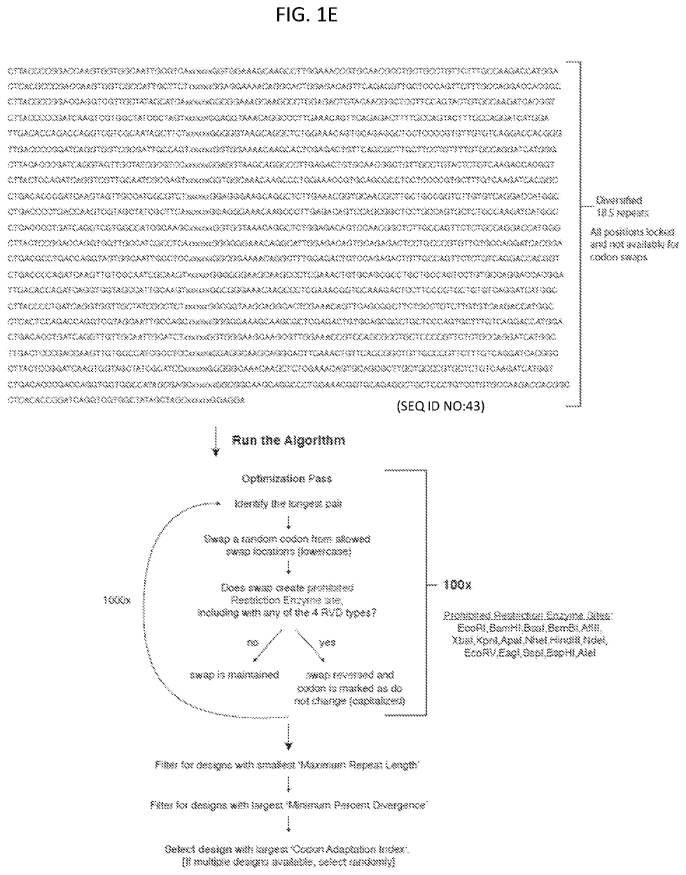

For example, the initial nucleic acid sequence may encode a DBP comprising 18.5 TALE RUs, where each RU comprises the amino acid sequence LTPDQVVAIAS X₁₂X₁₃GGKQALETVQRLLPVL QDHG (SEQ ID NO:1), where X₁₂X₁₃ may be RVDs. In some embodiments, the RVDs may be the same or may be specified to be identical to exemplify a scenario where the algorithm cannot count on the different RVDs decreasing the length of identical sequences. Such a nucleic acid sequence is exemplified in FIG. 1A. The depicted nucleic acid sequence (SEQ ID NO:40) encodes 18.5 RUs having the same RVDs HD. Since the RUs are identical, there are 18 contiguous stretches of nts that are identical across the entire length (encoding the identical RUs) and 19 stretches of nucleotides that are identical over a length of 125 nts (the stretch of stretches of nucleotides encoding the half-RU is 125 nts long and is identical to the first 125 nts in each of the stretches of nts encoding each RU). A schematic of the DBP encoded by the nucleic acid sequence is depicted where the identical rectangles represent RUs, the last rectangle represents the half-RU. An N-cap region denoted by “N” and a C-cap region denoted by “C” are also shown. However, since the N-cap and C-cap regions do not include repeated sequences, the nucleic acid sequence encoding these regions need not be diversified. As depicted in FIG. 1A, the nucleic acid sequences is analyzed to identify longest pairs of nts sequences that are identical; and a codon is swapped at random; if the swap does not create a RE site (e.g., a recognition site for EcoRI, BamHI, BsaI, BsmBI, AflII, XbaI, KpnI, ApaI, NheI, HindIII, NdeI, EcoRV, EagI, SspI, BspHI, or AleI), the swap is retained and if the swap created a RE site, the codon is marked as non-replaceable. The steps of identification of longest pair of identical sequences and codon swap are performed about 1000 times until no pair of identical sequences that are longer than 14 nts are present. In addition, the steps may be performed simultaneously on multiple copies of the initial nucleic acid sequence.

The diversification method may yield multiple different diversified nucleic acid sequences which may be filtered based on shortest-longest identical pairs of sequences; minimum-percent-divergence, and/or codon adaptation index.

In certain embodiments, the method depicted in FIG. 1A may yield one or more nucleic acid sequences comprising 18 different stretches of nucleotides that each encode the same RU (identical sequence) and a 19^{th} stretch of nucleotides that encodes a half RU comprising the RVD, HD. Also depicted in FIG. 1A are nucleotides in bold which nts are not swapped as they serve as cloning sites.

Similarly, the method may be performed to generate a diversified nucleic acid sequence that encodes a DBP comprising 18.5RUs where each RU includes the same RVD, NG; to generate a diversified nucleic acid sequence that encodes a DBP comprising 18.5RUs where each RU includes the same RVD, NG; and generate a diversified nucleic acid sequence that encodes a DBP comprising 18.5RUs where each RU includes the same RVD, NI.

These diversified nucleic acid sequences can be used to assemble a DBP that comprises RUs with different RVDs, where the stretches of nucleic acid sequence encoding the RUs having different RVDs are derived from the diversified nucleic acid sequences. Exemplary embodiments of methods for diversifying a nucleic acid sequence are depicted in FIGS. 1B-IE. FIG. 1B shows a design and diversification scheme for the core 18.5 RU modular diversified repeat TALE which may be used in an assembly pipeline for assembling a DBP. In this embodiment, codons (“CACGAT”) encoding RVDs are locked during the initial diversification to encompass a scenario where all RVD positions are identical. The algorithm will produce the maximum diversity in the surrounding sequences. The algorithm then processes this sequence with the same method as described herein, adding in one extra step to ensure that any codon swap does not create a restriction site with any of the possible RVDs (NG, NH, NI, HD). FIG. 1C depicts another embodiment in which certain modifications were made to the diversification method. (A) First, to extend the possible size range of diversified repeat TALEs from 18.5 RUs to 21.5 RUs, while keeping the maximum diversity within the previously designed 18.5 RUs, an additional nucleic acid sequence encoding 3 RUs were added onto the beginning of the previously selected 18.5 RUs. The sequence, in which only lower case codons can be swapped, was again ran through the algorithm to diversify the first the RUs as much as possible within the context of the already experimentally validated 18.5 RU design. The 3 RUs are added onto the design instead of creating a new 21.5 RU de novo to keep maximum diversity within the shorter TALE designs to ensure there would be no recombination by lentivirus within these shorter designs, as was experimentally validated, as these designs may be more commonly used for screens and therapeutic purposes. (B) Second, sticky ends (shown by underlining) for cloning ligation sites were made to be compatible with the earlier version of the TALE Assembly Pipeline (FIG. 1A). Some small changes were made by hand to introduce sticky ends not present and ensure that the longest repeat length did not increase. (C) The full 21.5 RU design with all HD RVDs was modified to allow Quality Control (QC) checks of the assembled designs to ensure the correct TALE sequence was assembled. AleI sites (dotted line) and BspHI sites were introduced to locations where the longest repeat length would not be increased. These restriction sites are used for QC when assembled into the standard destination vectors or the lentiviral destination vectors, respectively. (D) The sequence context (with NG RVDs) the additional first 3 RUs were diversified within. FIG. 1E. Further diversification made on NG and NH RVDs within the final 21.5 RU design. The algorithm was ran again on two sequences, in which the pink xxxxxx was either aacggt (NG) or aaccat (NH) at every position. This produced 4 final 21.5 RU designs with each containing the same RVD at every position—giving a NG, NH, NI, and HD final sequence. For assembly of a given TALE, the sequence of each needed RVD is taken from the correct position within that RVD's final sequence. For TALEs shorter than 21.5 RUs, a RU is successively removed from the start of the sequence.

The computer-implemented methods of the present disclosure may involve using one or more processors and one or more transitory or non-transitory computer-readable media comprising instructions stored thereon, which when executed by the one or more processors, cause the one or more processors to perform the steps for diversifying a nucleic acid sequence. The computer-implemented methods of the present disclosure may further comprise one or more steps that are not computer-implemented, e.g., generation of the diversified nucleic acid by e.g., cloning segments of nucleic acids; generation of a lentiviral vector comprising the diversified nucleic acid. In certain embodiments, the computer-implemented method may be performed on a local computer. In certain embodiments, the computer-implemented method may be performed on a remote computer, e.g., using a cloud-based computer that can intake an initial nucleic acid sequence and generate one or more diversified nucleic acid sequence that is provided to a user.

The nucleic acid sequence may be DNA or RNA. In certain embodiments, the nucleic acid sequence is DNA. In certain embodiments, the diversified nucleic acid sequence may be DNA or RNA. In certain embodiments, the diversified nucleic acid sequence is DNA. In certain embodiments, the nucleic acid sequence is DNA and the diversified nucleic acid sequence is DNA or RNA. In certain embodiments, the nucleic acid sequence is RNA and the diversified nucleic acid sequence is DNA or RNA.

The DBP may be any protein known to specifically bind to a nucleotide sequence and may include TALE DBPs, ZFP, megaTALs, etc. The TALE DBPs may include RUs and N-cap and C-cap regions from TALE proteins from Xanthomonas, Ralstonia, Burkholderia, or Paraburkholderia.

The diversified nucleic acid sequence may be present in a viral vector, e.g., a lentiviral vector for delivery to a host cell. In certain aspects, the host cell is a human cell, in vivo or ex vivo. The human cell may be diseased cell such as a cancer cell. The human cell may be a stem cell, a hematopoietic stem cell, or the like.

In examples where the RUs are derived from RUs of TALE proteins, X₁₋₁₁ is at least 80% identical, at least 90% identical, or 100% identical to LTPEQVVAIAS (SEQ ID NO: 6). X_{14-20 or 21 or 22} is at least 80% identical to GGRPALE (SEQ ID NO: 44).

The RUs may each comprise 33-36 amino acid long sequence having at least 80% sequence identity to the amino acid sequence:

LTPDQVVAIAS X₁₂X₁₃GGKQALETVQRLLPVL QDHG (SEQ ID NO: 1), or having the sequence of SEQ ID NO:1 with one or more conservative amino acid substitutions thereto; wherein X₁₂X₁₃ is HH, KH, NH, NK, NQ, RH, RN, SS, NN, SN, KN, NI, KI, RI, HI, SI, NG, HG, KG, RG, RD, SD, HD, ND, KD, YG, YK, NV, HN, H*, HA, KA, N*, NA, NC, NS, RA, CI, or S*, where (*) means X₁₃ is absent.

In certain aspects, the RUs may comprise a 33-36 amino acid long sequence having a sequence at least 85%, at least 90%, at least 91%, at least 92%, at least 93%, at least 94%, at least 95%, or more, or identical to SEQ ID NO: 1.

In certain aspects, the RUs and the half-RU are derived from Xanthomonas TALE. In certain aspects, X₁₋₁₁ is at least 80%, at least 90%, or 100% identical to LTPEQVVAIAS (SEQ ID NO: 6), LTPAQVVAIAS (SEQ ID NO: 9), LTPDQVVAIAN (SEQ ID NO: 10), LTPDQVVAIAS (SEQ ID NO: 11), LTPYQVVAIAS (SEQ ID NO: 12), LTREQVVAIAS (SEQ ID NO: 13), or LSTAQVVAIAS (SEQ ID NO: 14).

In certain aspects, X_{14-20 or 21 or 22} is at least 80%, at least 90%, at least 95%, or 100% identical to GGKQALETVQRLLPVLCQDHG (SEQ ID NO: 15), GGKQALATVQRLLPVLCQDHG (SEQ ID NO: 16), GGKQALETVQRVLPVLCQDHG (SEQ ID NO: 17), or GGKQALETVQRVLPVLCQDHG (SEQ ID NO: 17).

In certain aspects, the DBP may include a plurality of RUs ordered from N-terminus to C-terminus of the DBP. For example, the DBP may include 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 RUs. In certain aspects, the DBP may include a plurality of RUs of naturally occurring transcription activator like effector (TALE) proteins, such as RUs from Xanthomonas or Ralstonia TALE proteins.

In certain aspects, one or more RUs in a DBP may be at least 80%, 85%, 90%, 91%, 92%, 93%, 94%, 95%, 96%, 97%, 98%, or 99%, or a 100% identical to a RU provided herein. Percent identity between a pair of sequences may be calculated by multiplying the number of matches in the pair by 100 and dividing by the length of the aligned region, including gaps. Identity scoring only counts perfect matches and does not consider the degree of similarity of amino acids to one another. Only internal gaps are included in the length, not gaps at the sequence ends.

$$Percent Identity = ( Matches \times 100 ) / Length of aligned region ( with gaps )$$

The phrase “conservative amino acid substitution” refers to substitution of amino acid residues within the following groups: 1) L, I, M, V, F; 2)R, K; 3) F, Y, H, W, R; 4) G, A, T, S; 5) Q, N; and 6) D, E. Conservative amino acid substitutions may preserve the activity of the protein by replacing an amino acid(s) in the protein with an amino acid with a side chain of similar acidity, basicity, charge, polarity, or size of the side chain.

Guidance for substitutions, insertions, or deletions may be based on alignments of amino acid sequences of proteins from different species or from a consensus sequence based on a plurality of proteins having the same or similar function.

In certain aspects, the disclosed DBP may include a nuclear localization sequence (NLS) to facilitate entry into an organelle of a cell, e.g., the nucleus of a cell, e.g., an animal or a plant cell. In certain aspects, the disclosed DBP may include a half-RU or a partial RU that is 15-20 amino acid long sequence. Such a half-RU may be included after the last RU present in the DBP and may be derived from a RU identified in Xanthomonas or Ralstonia TALE protein.

The disclosed DBP may include an N-terminus region. The N-terminus region may be the N-cap domain or a fragment thereof from TALE proteins like those expressed in Burkholderia, Paraburkholderia, or Xanthomonas. In certain aspects, the disclosed DBP may include a C-terminus region. The C-terminus region may be a C-cap domain or a fragment thereof from TALE proteins like those expressed in Burkholderia, Paraburkholderia, or Xanthomonas.

In certain aspects, the N-terminus region may be at least 80%, 85%, 90%, 91%, 92%, 93%, 94%, 95%, 96%, 97%, 98%, or 99%, or a 100% identical to the N-terminus region sequence provided in Table 2. This amino acid sequence includes a M added to the N-terminus which is not present in the wild type N-cap region of a Xanthomonas TALE protein. This amino acid sequence is generated by deleting amino acids N+288 through N+137 of the N-terminus region of a TALE protein, adding a M, such that amino acids N+136 through N+1 of the N-terminus region of the TALE protein are present.

In some aspects, the N-terminus region can be a truncated version of the wild type N-cap region such that the N-terminus region includes amino acids from position 1 (N) through position 120 (K) of the naturally occurring Xanthomonas spp.-N-cap region and has the follow sequence:

(SEQ ID NO: 18)
KPKVRSTVAQHHEALVGHGFTHAHIVALSQHPAALGTVAVKYQDMIAAL
PEATHEAIVGVGKQWSGARALEALLTVAGELRGPPLQLDTGQLLKIAKR
GGVTAVEAVHAWRNALTGAPLN.

In some aspects, the N-cap region can be truncated such that the N-terminus region includes amino acids from position 1 (N) through position 115 (S) of the naturally occurring Xanthomonas spp.-N-cap region and has the follow sequence:

(SEQ ID NO: 19)
STVAQHHEALVGHGFTHAHIVALSQHPAALGTVAVKYQDMIAALPEATH
EAIVGVGKQWSGARALEALLTVAGELRGPPLQLDTGQLLKIAKRGGVTA
VEAVHAWRNALTGAPLN.

In some aspects, the N-cap region can be truncated such that the N-terminus region includes amino acids from position 1 (N) through position 110 (H) of the naturally occurring Xanthomonas spp.-N-cap region and has the follow sequence:

(SEQ ID NO: 20)
HHEALVGHGFTHAHIVALSQHPAALGTVAVKYQDMIAALPEATHEAIVG
VGKQWSGARALEALLTVAGELRGPPLQLDTGQLLKIAKRGGVTAVEAVH
AWRNALTGAPLN.

In certain aspects, the DBP may include a C-terminus region at C-terminus of the recombinant polypeptide which C-terminus region is derived from the C-cap region of a Xanthomonas TALE protein and follows the last half-RU. In certain aspects, the C-terminus region at the C-terminus may be linked to the last half-RU via a linker.

In certain aspects, the C-terminus region may be at least 80%, 85%, 90%, 91%, 92%, 93%, 94%, 95%, 96%, 97%, 98%, or 99%, or a 100% identical to the C-terminus region sequence provided in Table 2.

In certain aspects, the RUs are derived from Xanthomonas TALEs. In plant genomes, such as Xanthomonas, the natural TALE-binding sites begin with a thymine (T), which may be specified by a cryptic signal within the non-repetitive N-terminus region of the TALE polypeptide; in some cases this region may be referred to as repeat 0. In animal genomes, TALE binding sites do not necessarily have to begin with a thymine (T) and recombinant DBP disclosed herein may target DNA sequences that begin with T, A, G or C. In certain aspects, the recombinant DBP disclosed herein may target DNA sequences that begin with T. The tandem repeat of TALE RUs ends with a half-length repeat or a stretch of sequence that may share identity with only the first 20 amino acids of a repetitive full length TALE RU and this half repeat may be referred to as a half-monomer, a half RU, or a half repeat. Therefore, it follows that the length of the DNA sequence being targeted by DBP derived from TALEs is equal to the number of full RUs plus two. Thus, for example, DBP may be engineered to include X number (e.g., 9, 10, 11, 12, 13, 14, 15, 16, 17, 18, 19, 20, 21, 22, 23, 24, 25, or 26) full length RUs that are specifically ordered or arranged to target nucleic acid sequences of X+2 length (e.g., 11, 12, 13, 14, 15, 16, 17, 18, 19, 20, 21, 22, 23, 24, 25, 26, 27, or 28 nucleotides, respectively), with the N-terminus region binding “T” and the last RU being a half-repeat.

In certain aspects, a Xanthomonas spp.-derived repeat units can have a sequence of LTPDQVVAIASNHGGKQALETVQRLLPVLCQDHG (SEQ ID NO: 21) comprising an RVD of NH, which recognizes guanine. A Xanthomonas spp.-derived repeat units can have a sequence of LTPDQVVAIASNGGGKQALETVQRLLPVLCQDHG (SEQ ID NO: 22) comprising an RVD of NG, which recognizes thymidine. A Xanthomonas spp.-derived repeat units can have a sequence of LTPDQVVAIASNIGGKQALETVQRLLPVLCQDHG (SEQ ID NO: 23) comprising an RVD of NI, which recognizes adenosine. A Xanthomonas spp.-derived repeat units can have a sequence of LTPDQVVAIASHDGGKQALETVQRLLPVLCQDHG (SEQ ID NO: 24) comprising an RVD of HD, which recognizes cytosine.

In certain aspects, the RUs and one or both N-terminus and C-terminus regions may be derived from a transcription activator like effector-like protein (TALE-like protein) of Ralstonia solanacearum. Repeat units derived from Ralstonia solanacearum can be 33-35 amino acid residues in length. In some aspects, the repeat can be derived from the naturally occurring Ralstonia solanacearum TALE-like protein.

As noted herein, the RUs may have the sequence X₁₋₁₁X₁₂X₁₃X_{14-33, 34, or 35} (SEQ ID NO: 4), where X₁₋₁₁ is a chain of 11 contiguous amino acids, X_{14-33 or 34 or 35} is a chain of 20, 21 or 22 contiguous amino acids, X₁₂X₁₃ is RVD and is selected from: (a) NH, HH, KH, NK, NQ, RH, RN, SS, NN, SN, or KN for recognition of guanine (G); (b) NI, KI, RI, HI, or SI for recognition of adenine (A); (c) NG, HG, KG, or RG for recognition of thymine (T); (d) HD, RD, SD, ND, KD, or YG for recognition of cytosine (C); and (e) NV or HN for recognition of A or G; and (f) H*, HA, KA, N*, NA, NC, NS, RA, or S*for recognition of A or T or G or C, wherein (*) means that the amino acid at X₁₃ is absent. In certain aspects, X₁₋₁₁ may include a stretch of amino acids at least 80%, at least 90%, or a 100% identical to the X₁₋₁₁ residues of the following RUs from Ralstonia. In certain aspects, X_{14-33 or 34 or 35} may include a stretch of 20, 21, or 22 amino acids at least 80%, at least 90%, or a 100% identical to the X_{14-33 or 34 or 35} residues of the following RUs from Ralstonia: LDTEQVVAIASHNGGKQALEAVKADLLDLLGAPYV (SEQ ID NO: 26), LNTEQVVAVASNKGGKQALEAVGAQLLALRAVPYE (SEQ ID NO: 27), LSTAQVAAIASHDGGKQALEAVGTQLVVLRAAPYA (SEQ ID NO: 28), LSTAQVVAVAGRNGGKQALEAVRAQLPALRAAPYG (SEQ ID NO: 29), or LSTAQVVAVASSNGGKQALEAVWALLPVLRATPYD (SEQ ID NO: 30).

In certain aspects, a Ralstonia solanacearum- repeat unit can have at least 80% sequence identity with any one of the Ralstonia RUs provided herein.

In certain aspects, the DBP may include a N-cap region at the N-terminus which may be present immediately adjacent the first RU or may be linked to the first RU via a linker. In some aspects, an DBP of the present disclosure can have the full length naturally occurring N-terminus of a naturally occurring Ralstonia solanacearum-derived protein. In some aspects, any truncation of the full length naturally occurring N-terminus of a naturally occurring Ralstonia solanacearum-derived protein can be used at the N-terminus of a DBP of the present disclosure. For example, in some aspects, amino acid residues at positions 1 (H) to position 137 (F) of the naturally occurring Ralstonia solanacearum-derived protein N-terminus can be used as the N-cap region. In particular aspects, the truncated N-terminus from position 1 (H) to position 137 (F) can have a sequence as follows:

FGKLVALGYSREQIRKLKQESLSEIAKYHTTLTGQGFTHADICRISRRRQSLRVVARNYP ELAAALPELTRAHIVDIARQRSGDLALQALLPVATALTAAPLRLSASQIATVAQYGERP AIQALYRLRRKLTRAPLH (SEQ ID NO: 31). In some aspects, the naturally occurring N-terminus of Ralstonia solanacearum can be truncated to any length and used as the N-cap of the engineered DNA binding polypeptide. For example, the naturally occurring N-terminus of Ralstonia solanacearum can be truncated to include amino acid residues at position 1 (H) to position 120 (K) as follows:
KQESLSEIAKYHTTLTGQGFTHADICRISRRRQSLRVVARNYPELAAALPELTRAHIVDI ARQRSGDLALQALLPVATALTAAPLRLSASQIATVAQYGERPAIQALYRLRRKLTRAPL H (SEQ ID NO: 32) and used as the N-cap of the DBP. The naturally occurring N-terminus of Ralstonia solanacearum can be truncated amino acid residues to include positions 1 to 115 and used at the N-cap of the engineered DNA binding domain. The naturally occurring N-terminus of Ralstonia solanacearum can be truncated to amino acid residues at positions 1 to 50, 1 to 70, 1 to 100, 1 to 120, 1 to 130, 10 to 40, 60 to 100, or 100 to 120 and used as the N-cap of the engineered DNA binding domain. As noted for N-cap region derived from Xanthomonas TALE, the amino acid residues are numbered backward from the first repeat unit such that the amino acid (H in this case) of the N-cap adjacent the first RU is numbered 1 while the N-terminal amino acid of the N-cap is numbered 137 (and is F in this case) or 120 (and is K in this case).

In some aspects, the N-cap, referred to as the amino terminus or the “NH2” domain, can recognize a guanine. In some aspects, the N-cap can be engineered to bind a cytosine, adenosine, thymidine, guanine, or uracil.

In some aspects, an DBP of the present disclosure can include a plurality of RUs followed by a final single half-repeat also derived from Ralstonia solanacearum. The half repeat can have 15 to 23 amino acid residues, for example, the half repeat can have 19 amino acid residues. In particular aspects, the half-repeat can have a sequence as follows: LSTAQVVAIACISGQQALE (SEQ ID NO: 33).

In some aspects, an DBP of the present disclosure can have the full length naturally occurring C-terminus of a naturally occurring Ralstonia solanacearum-derived protein as a C-cap region that is conjugated to the last RU. In some aspects, any truncation of the full length naturally occurring C-terminus of a naturally occurring Ralstonia solanacearum-derived protein can be used as the C-cap. For example, in some aspects, the DBP can comprise amino acid residues at position 1 (A) to position 63 (S) as follows:

AIEAHMPTLRQASHSLSPERVAAIACIGGRSAVEAVRQGLPVKAIRRIRREKAPVAGPPP AS (SEQ ID NO: 34) of the naturally occurring Ralstonia solanacearum-derived protein C-terminus. In some aspects, the naturally occurring C-terminus of Ralstonia solanacearum can be truncated to any length and used as the C-cap of the DBP. For example, the naturally occurring C-terminus of Ralstonia solanacearum can be truncated to amino acid residues at positions 1 to 63 and used as the C-terminus of the DBP. The naturally occurring C-terminus of Ralstonia solanacearum can be truncated amino acid residues at positions 1 to 50 and used as the C-cap of the DBP. The naturally occurring C-terminus of Rastonia solanacearum can be truncated to amino acid residues at positions 1 to 63, 1 to 50, 1 to 70, 1 to 100, 1 to 120, 1 to 130, 10 to 40, 60 to 100, or 100 to 120 and used as the C-cap of the DBP. Exemplary sequences of domains of a DBP as disclosed herein are as follows:

| Description | Sequence |
|---|---|
| Truncated N-terminus; | SEIAKYHTTLTGQGFTHADICRISRRRQSLRVVARNYPELAAALP |
| positions 1 (H) to 115 (S) of | ELTRAHIVDIARQRSGDLALQALLPVATALTAAPLRLSASQIATV |
| the naturally occurring | AQYGERPAIQALYRLRRKLTRAPLH (SEQ ID NO: 35) |
| Ralstonia solanacearum- | |
| derived protein N-terminus | |
| Truncated N-terminus; | FGKLVALGYSREQIRKLKQESLSEIAKYHTTLTGQGFTHADICRI |
| positions 1 (H) to 137 (F) of | SRRRQSLRVVARNYPELAAALPELTRAHIVDIARQRSGDLALQA |
| the naturally occurring | LLPVATALTAAPLRLSASQIATVAQYGERPAIQALYRLRRKLTR |
| Ralstonia solanacearum- | APLH (SEQ ID NO: 31) |
| derived protein N-terminus | |
| Truncated N-terminus; | KQESLSEIAKYHTTLTGQGFTHADICRISRRRQSLRVVARNYPEL |
| positions 1 (H) to 120 (K) of | AAALPELTRAHIVDIARQRSGDLALQALLPVATALTAAPLRLSAS |
| the naturally occurring | QIATVAQYGERPAIQALYRLRRKLTRAPLH (SEQ ID NO: 32) |
| Ralstonia solanacearum- | |
| derived protein N-terminus | |
| Half-repeat | LSTAQVVAIACISGQQALE (SEQ ID NO: 33) |
| Truncated C-terminus; | AIEAHMPTLRQASHSLSPERVAAIACIGGRSAVEAVRQGLPVKAI |
| positions 1 (A) to 63 (S) of the | RRIRREKAPVAGPPPAS (SEQ ID NO: 34) |
| naturally occurring Ralstonia | |
| solanacearum-derived protein | |
| C-terminus | |

DBP Derived from Animal Pathogens

In some aspects, the present disclosure provides DNA binding polypeptide in which the repeat units can be derived from a Legionellales bacterium, a species of the genus of Legionella, such as L. quateirensis or L. maceachernii, the genus of Burkholderia, the genus of Paraburkholderia, or the genus of Francisella.

### Functional Domains

A DBP as disclosed herein can be associated with a functional domain as described in the preceding sections. The functional domain can provide different types of activity, such as genome editing, gene regulation (e.g., activation or repression), or visualization of a genomic locus via imaging. In certain aspects, the functional domain is heterologous to the DBP. Heterologous in the context of a functional domain and a DBP as used herein indicates that these domains are derived from different sources and do not exist together in nature. In some aspects, the nuclease can be a cleavage half domain, which dimerizes to form an active full domain capable of cleaving DNA. In other aspects, the nuclease can be a cleavage domain, which is capable of cleaving DNA without needing to dimerize. For example, a nuclease comprising a cleavage half domain can be an endonuclease, such as FokI or Bfil. In some aspects, two cleavage half domains (e.g., FokI or Bfil) can be fused together to form a fully functional single cleavage domain.

A nuclease domain fused to a DBP can be an endonuclease or an exonuclease. An endonuclease can include restriction endonucleases and homing endonucleases. An endonuclease can also include S1 Nuclease, mung bean nuclease, pancreatic DNase I, micrococcal nuclease, or yeast HO endonuclease. An exonuclease can include a 3′-5′ exonuclease or a 5′-3′ exonuclease. An exonuclease can also include a DNA exonuclease or an RNA exonuclease. Examples of exonuclease includes exonucleases I, II, III, IV, V, and VIII; DNA polymerase I, RNA exonuclease 2, and the like.

A nuclease domain fused to a DBP as disclosed herein can be a restriction endonuclease (or restriction enzyme). In some instances, a restriction enzyme cleaves DNA at a site removed from the recognition site and has a separate binding and cleavage domains. In some instances, such a restriction enzyme is a Type IIS restriction enzyme.

As another example, DBP as disclosed herein can be linked to a gene regulating domain. A gene regulation domain can be an activator or a repressor. For example, a DBP as disclosed herein can be linked to an activation domain, such as VP16, VP64, p65, p300 catalytic domain, TETI catalytic domain, TDG, Ldbl self-associated domain, SAM activator (VP64, p65, HSF1), or VPR (VP64, p65, Rta). Alternatively, a DBP can be linked to a repressor, such as KRAB, Sin3a, LSD1, SUV39H1, G9A (EHMT2), DNMT1, DNMT3A-DNMT3L, DNMT3B, KOX, TGF-beta-inducible early gene (TIEG), v-erbA, SID, MBD2, MBD3, Rb, or MeCP2. The terms “repressor,” “repressor domain,” and “transcriptional repressor” are used herein interchangeably to refer to a polypeptide that decreases expression of a gene.

In some aspects, a DBP as disclosed herein can be linked to a DNA modifying protein, such as DNMT3a. A DBP can be linked to a chromatin-modifying protein, such as lysine-specific histone demethylase 1 (LSD1). A DBP can be linked to a protein that is capable of recruiting other proteins, such as KRAB. The DNA modifying protein (e.g., DNMT3a) and proteins capable of recruiting other proteins (e.g., KRAB) can serve as repressors of transcription. Thus, DBP linked to a DNA modifying protein (e.g., DNMT3a) or a domain capable of recruiting other proteins (e.g., KRAB, a domain found in transcriptional repressors, such as Kox1) can provide gene repression functionality, can serve as transcription factors, wherein the DBP provides specificity and targeting and the DNA modifying protein and the protein capable of recruiting other proteins provides gene repression functionality, which can be referred to as an engineered genomic regulatory complex or a DBP-gene regulator (DBP-GR) and, more specifically, as a DBP -transcription factor (DBP-TF).

In certain aspects, the functional domain may be an imaging domain, e.g., a fluorescent protein, biotinylation reagent, tag (e.g., 6X-His or HA). A DBP can be linked to a fluorophore, such as Hydroxycoumarin, methoxycoumarin, Alexa fluor, aminocoumarin, Cy2, FAM, Alexa fluor 488, Fluorescein FITC, Alexa fluor 430, Alexa fluor 532, HEX, Cy3, TRITC, Alexa fluor 546, Alexa fluor 555, R-phycoerythrin (PE), Rhodamine Red-X, Tamara, Cy3.5, Rox, Alexa fluor 568, Red 613, Texas Red, Alexa fluor 594, Alexa fluor 633, Allophycocyanin, Alexa fluor 633, Cy5, Alexa fluor 660, Cy5.5, TruRed, Alexa fluor 680, Cy7, GFP, or mCHERRY.

In certain aspects, the DBP is not fused with a functional domain having a genome modifying activity, such as, cleavage activity, DNA methylation activity, chromatin-modifying protein, transcriptional activation, or transcriptional repression.

### Targets

In some aspects, a cell that expresses the DBP disclosed herein may be a mammalian cell such as a stem cell (e.g., human embryonic stem cell or induced pluripotent stem cell), human hematopoietic stem cell “HSC” (e.g., CD34+ HSC), hematopoietic progenitor cell (HPC), a cell in the erythroid lineage, a lymphocyte, a T-cell, CAR-T cells, a cancer cell, ex vivo cell, etc. A cell may be selected for stable expression of the DBP. A cell may be selected for expressing a threshold level of the DBP. A cell selected for expression of the DBP may be subjected to expansion, freeze/thaw or otherwise prepared for introduction into a subject in need thereof.

A cell expressing a DBP either constitutively or in an inducible manner may be administered to the subject, for instance in the circulatory system by means of intravenous delivery or delivery into a solid tissue such as bone marrow.

Exemplary mammalian cells can include, but are not limited to, 293A cell line, 293FT cell line, 293F cells, 293 H cells, HEK 293 cells, CHO DG44 cells, CHO-S cells, CHO-K1 cells, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, T-REx™-HeLa cell line, NC-HIMT cell line, PC12 cell line, primary cells (e.g., from a human) including primary T cells, primary hematopoietic stem cells, primary human embryonic stem cells (hESCs), and primary induced pluripotent stem cells (iPSCs). In some cases, the cell may be a human erythroid progenitor or precursor cells. The erythroid progenitor or precursor cells may be autologous.

In some cases, a target cell is a cancerous cell. Cancer can be a solid tumor or a hematologic malignancy. The solid tumor can include a sarcoma or a carcinoma. Exemplary sarcoma target cell can include, but are not limited to, cell obtained from alveolar rhabdomyosarcoma, alveolar soft part sarcoma, ameloblastoma, angiosarcoma, chondrosarcoma, chordoma, clear cell sarcoma of soft tissue, dedifferentiated liposarcoma, desmoid, desmoplastic small round cell tumor, embryonal rhabdomyosarcoma, epithelioid fibrosarcoma, epithelioid hemangioendothelioma, epithelioid sarcoma, esthesioneuroblastoma, Ewing sarcoma, extrarenal rhabdoid tumor, extraskeletal myxoid chondrosarcoma, extraskeletal osteosarcoma, fibrosarcoma, giant cell tumor, hemangiopericytoma, infantile fibrosarcoma, inflammatory myofibroblastic tumor, Kaposi sarcoma, leiomyosarcoma of bone, liposarcoma, liposarcoma of bone, malignant fibrous histiocytoma (MFH), malignant fibrous histiocytoma (MFH) of bone, malignant mesenchymoma, malignant peripheral nerve sheath tumor, mesenchymal chondrosarcoma, myxofibrosarcoma, myxoid liposarcoma, myxoinflammatory fibroblastic sarcoma, neoplasms with perivascular epitheioid cell differentiation, osteosarcoma, parosteal osteosarcoma, neoplasm with perivascular epitheioid cell differentiation, periosteal osteosarcoma, pleomorphic liposarcoma, pleomorphic rhabdomyosarcoma, PNET/extraskeletal Ewing tumor, rhabdomyosarcoma, round cell liposarcoma, small cell osteosarcoma, solitary fibrous tumor, synovial sarcoma, or telangiectatic osteosarcoma.

Exemplary carcinoma target cell can include, but are not limited to, cell obtained from anal cancer, appendix cancer, bile duct cancer (i.e., cholangiocarcinoma), bladder cancer, brain tumor, breast cancer, cervical cancer, colon cancer, cancer of Unknown Primary (CUP), esophageal cancer, eye cancer, fallopian tube cancer, gastroenterological cancer, kidney cancer, liver cancer, lung cancer, medulloblastoma, melanoma, oral cancer, ovarian cancer, pancreatic cancer, parathyroid disease, penile cancer, pituitary tumor, prostate cancer, rectal cancer, skin cancer, stomach cancer, testicular cancer, throat cancer, thyroid cancer, uterine cancer, vaginal cancer, or vulvar cancer.

Alternatively, the cancerous cell can comprise cells obtained from a hematologic malignancy. Hematologic malignancy can comprise a leukemia, a lymphoma, a myeloma, a non-Hodgkin's lymphoma, or a Hodgkin's lymphoma. In some cases, the hematologic malignancy can be a T-cell based hematologic malignancy. Other times, the hematologic malignancy can be a B-cell based hematologic malignancy. Exemplary B-cell based hematologic malignancy can include, but are not limited to, chronic lymphocytic leukemia (CLL), small lymphocytic lymphoma (SLL), high-risk CLL, a non-CLL/SLL lymphoma, prolymphocytic leukemia (PLL), follicular lymphoma (FL), diffuse large B-cell lymphoma (DLBCL), mantle cell lymphoma (MCL), Waldenstrom's macroglobulinemia, multiple myeloma, extranodal marginal zone B cell lymphoma, nodal marginal zone B cell lymphoma, Burkitt's lymphoma, non-Burkitt high grade B cell lymphoma, primary mediastinal B-cell lymphoma (PMBL), immunoblastic large cell lymphoma, precursor B-lymphoblastic lymphoma, B cell prolymphocytic leukemia, lymphoplasmacytic lymphoma, splenic marginal zone lymphoma, plasma cell myeloma, plasmacytoma, mediastinal (thymic) large B cell lymphoma, intravascular large B cell lymphoma, primary effusion lymphoma, or lymphomatoid granulomatosis. Exemplary T-cell based hematologic malignancy can include, but are not limited to, peripheral T-cell lymphoma not otherwise specified (PTCL-NOS), anaplastic large cell lymphoma, angioimmunoblastic lymphoma, cutaneous T-cell lymphoma, adult T-cell leukemia/lymphoma (ATLL), blastic NK-cell lymphoma, enteropathy-type T-cell lymphoma, hematosplenic gamma-delta T-cell lymphoma, lymphoblastic lymphoma, nasal NK/T-cell lymphomas, or treatment-related T-cell lymphomas.

In some cases, a cell can be a tumor cell line. Exemplary tumor cell line can include, but are not limited to, 600MPE, AU565, BT-20, BT-474, BT-483, BT-549, Evsa-T, Hs578T, MCF-7, MDA-MB-231, SkBr3, T-47D, HeLa, DU145, PC3, LNCaP, A549, H1299, NCI-H460, A2780, SKOV-3/Luc, Neuro2a, RKO, RKO-AS45-1, HT-29, SW1417, SW948, DLD-1, SW480, Capan-1, MC/9, B72.3, B25.2, B6.2, B38.1, DMS 153, SU.86.86, SNU-182, SNU-423, SNU-449, SNU-475, SNU-387, Hs 817.T, LMH, LMH/2A, SNU-398, PLHC-1, HepG2/SF, OCI-Ly1, OCI-Ly2, OCI-Ly3, OCI-Ly4, OCI-Ly6, OCI-Ly7, OCI-Ly10, OCI-Ly18, OCI-Ly19, U2932, DB, HBL-1, RIVA, SUDHL2, TMD8, MEC1, MEC2, 8E5, CCRF-CEM, MOLT-3, TALL-104, AML-193, THP-1, BDCM, HL-60, Jurkat, RPMI 8226, MOLT-4, RS4, K-562, KASUMI-1, Daudi, GA-10, Raji, JeKo-1, NK-92, and Mino.

Genetic modification can involve introducing a functional gene for therapeutic purposes, knocking out a gene for therapeutic gene, or engineering a cell ex vivo (e.g., HSCs or CAR T cells) to be administered back into a subject in need thereof. Cells, such as hematopoietic stem cells (HSCs) and T cells, can be engineered ex vivo to express the DBP. Alternatively, nucleic acid encoding the DBP can be directly administered to a subject in need thereof.

The target gene may be an endogenous gene such as human fetal gamma globin gene, PDCD 1 gene, a CTLA4 gene, a LAG3 gene, a TET2 gene, a ETLA gene, a HA VCR2 gene, a CCR5 gene, a CXCR4 gene, a TRA gene, a TRE gene, a E2M gene, an albumin gene, a HEE gene, a HEAl gene, a TTR gene, a NR3C1 gene, a CD52 gene, an erythroid specific enhancer of the ECLllA gene, a CELE gene, a TGFER1 gene, a SERPINA1 gene, a HEV genomic DNA in infected cells, a CEP290 gene, a DMD gene, a CFTR gene, or an IL2RG gene.

### EXAMPLES

As can be appreciated from the disclosure provided above, the present disclosure has a wide variety of applications. Accordingly, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Those of skill in the art will readily recognize a variety of noncritical parameters that could be changed or modified to yield essentially similar results. Thus, the following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, dimensions, etc.) but some experimental errors and deviations should be accounted for.

### Materials and Methods

Cell culture. HUDEP-2 cells (PMID 23533656) were grown in StemSpan SFEM (Stemcell Technologies) supplemented with 2% PennStrep, 1% L-Glutamine, 1 μg/mL doxycycline, 100 ng/mL recombinant human SCF (Peprotech), 3 IU/mL recombinant human EPO (Peprotech), and 10-6 M dexamethasone.

TALE transfection. mRNA was in vitro transcribed using T7 mScript™ Standard mRNA Production System (Cellscript) following manufacturer's instructions and RNA size and concentrations were determined using the Advanced Analytical Fragment Analyzer (Agilent). 5×10⁵ HUDEP-2 cells were electroporated with 2 ug, 4ug, 5ug, or 10ug TALE mRNA and 100 ul BTXpress solution (BTX) in 96-well cuvette (BTX) using the ECM 830 Square Wave Electroporation System (Harvard Apparatus) and HT200 Plate Handler (BTX) with 250 mS interval, 250V for 5 msec pulse. Electroporated cells were transferred to 12 or 6-well plates, and RNA was harvested 12, 24, or 48 hours later. After the initial time course experiment, all samples were harvested at 48 hours.

Generation of Clonal Lines. 5×10⁶ HUDEP-2 cells were transfected with TALEN pairs recognizing the AAVS1 safe harbor locus (5′-TTTCTGTCACCAATCCT-3′ (SEQ ID NO:2) and 5′-TCCCCTCCACCCCACAGT-3′ (SEQ ID NO:3); 2.5 ug mRNA per TALEN monomer), together with 2.5 ug AAVS1 donor plasmid using the Amaxa Human CD34+ Cell Nucleofector Kit (Lonza) on an Amaxa Nucleofector II Device (Lonza). Donor plasmid contains a splice acceptor site followed by T2A and GFP to utilize an endogenous promoter to drive expression of GFP after integration and an EFIalpha-driven TALE followed by WPRE (sequences provided below). Cells were maintained in a T-25 flask and media was changed every 24 hours post-transfection for two days. Approximately 7-12 days post-transfection, single cells were sorted for GFP expression into 96-well plates using the MoFlo Astrios (Beckman Coulter). Cells were maintained in 96-well plates for −12 days with doxycycline supplemented every two days and media changes on days 8 and 10. Wells containing cells were identified, consolidated, and further maintained.

Globin TaqMan qPCR. Total RNA was extracted from 2-5×10⁵ HUDEP-2 cells from clonal lines expressing stably-integrated TALEs or WT cells transfected with TALE mRNA using the RNeasy Micro Kit (Qiagen), and 100ng RNA was reverse transcribed with iScript Reverse Transcription Supermix (Bio-Rad). qPCRs were run with multiplexed Taqman primer/probe sets specific to HBG (Hs00361131_g1, VIC) and HBB (Hs00747223_g1, FAM) from ThermoFisher Scientific, cDNA diluted at a 1:10 ratio, and SsoAdvanced Universal Probes Supermix (Bio-Rad) on a Bio-Rad CFX96 Real Time System. Primer/probe sets were confirmed to be accurate and robust in multiplexing assays as no difference was observed when tested individually or multiplexed. In TALE transfection experiments, HBG measurements were normalized to measurements of HBB and compared to transfected control cells in biological triplicate. For analysis of stably-integrated TALE HUDEP-2 cell lines, amount of HBG expression was normalized to total HBB and HBG expression, with 2-5 measurements taken at different timepoints in culture per sample.

FLAG Immunofluorescence. In a 24-well poly-1-lysine coated cover glass bottom plate (BioMedTech Laboratories, Inc.), 7.5×10⁴ cells were deposited in a 30 ul droplet to the center of the well and allowed to settle for 40 minutes. Cells were fixed with 4% PFA (Polysciences Inc, #18814-10) for 10 minutes at room temperature, washed 3 times for 3 minutes each with PBS, permeabilized with 0.25% Triton (Triton X-100) in PBS for 10 minutes, and washed 3 times for 5 minutes each with PBS. Cover glass bottoms were incubated with blocking solution (2% BSA-PBS (Jackson Immunoresearch, #001-000-161)) for 45 minutes, then incubated with a 1:500 dilution of primary mouse monoclonal ANTI-FLAG® M2 antibody (F1804; Sigma) in 2% BSA-PBS for 2 hours, washed 3 times for 3 minutes each with 0.05% Tween (Bio-rad, #161-0781) in PBS, incubated with a 1:500 dilution of secondary antibody conjugated to either Cy3 or AlexaFluor-647 (Jackson Labs) for 1 hour, and washed 3 times for 5 minutes each with 0.05% Tween-PBS with the second wash containing 0.1 mg/mL DAPI solution (100 ng/mL in 1× PBS) and washing for 10 minutes. Lastly, cell samples in individual wells were mounted with 7-10 uL Prolong Gold (Molecular Probes P36930) antifade, sealed with 12 mm coverglasses (1.5, Electron Microscopy Sciences), and cured either overnight or for at least 2 hours prior to imaging. Samples were imaged using an inverted Nikon Eclipse Ti widefield microscope equipped with an Andor Zyla 4.2CL10 CMOS camera with a 4.2-megapixel sensor and 6.5 μm pixel size (18.8 mm diagonal FOV). Focused 3D cell images were acquired using a 40× 0.9 NA air objective. Acquired images were subject to 100 rounds of iterative blind deconvolution using Microvolution software (Microvolution, CA) to minimize the effect of out-of-focus blurring that is inherent to widefield microscopy optics. Deconvolved images were processed using in-house Matlab (version 2017B, Mathworks, Natick, MA) scripts to numerically estimate the average FLAG protein content in every cell nucleus, and for downstream statistical analysis.

Globin FACS. Approximately 1×10⁶ cells were harvested, fixed with 4% PFA for 15 minutes, permeabilized with acetone, incubated with 1:200 anti-HbF-APC (MHFH05; ThermoFisher) and 1:400 anti-Hemoglobin β (sc-21757; Santa Cruz) for at least 20 minutes at 4° C., rinsed with 0.5% BSA-PBS, and stained with DAPI for 15 minutes. FACS was run on a CytoFLEX S (Beckman Coulter).

HBG-A11 TALE protein binds to the sequence TATCCTCTTGGGGGCCCC (SEQ ID NO:4) in the γ-globin proximal promoter. HBG-A11 TALE protein includes N-cap (also called N-terminus region) and C-cap (also called C-terminus region) and RUs with RVDs as listed in Table 1.

TABLE 1
TALE Protein Domain Sequences

| TALE | | |
|---|---|---|
| Protein | | SEQ |
| Domain | Sequence | ID NO |
| N-terminus | MVDLRTLGYSQQQQEKIKPKVRSTVAQHHEALVGHGFT | 5 |
| region | HAHIVALSQHPAALGTVAVKYQDMIAALPEATHEAIVG | |
| | VGKQWSGARALEALLTVAGELRGPPLQLDTGQLLKIAK | |
| | RGGVTAVEAVHAWRNALTGA | |
| C-terminus | SIVAQLSRPDPALAALTNDHLVALACLGGRPALDAVKK | 7 |
| region | GLPHAPALIKRTNRRIPERTSHRVA | |
| NI RVD | LTPDQVVAIASNIGGKQALETVQRLLPVLCQDHG | 23 |
| (A) | | |
| NG RVD | LTPDQVVAIASNGGGKQALETVQRLLPVLCQDHG | 22 |
| (T) | | |
| HD RVD | LTPDQVVAIASHDGGKQALETVQRLLPVLCQDHG | 24 |
| (C) | | |
| NH RVD | LTPDQVVAIASNHGGKQALETVQRLLPVLCQDHG | 21 |
| (G) | | |
| NI last half | LTPDQVVAIASNIGGRPALE | 8 |
| repeat (A) | | |
| NG last | LTPDQVVAIASNGGGRPALE | 25 |
| half repeat | | |
| (T) | | |
| HD last | LTPDQVVAIASHDGGRPALE | 36 |
| half repeat | | |
| (C) | | |
| NH last | LTPDQVVAIASNHGGRPALE | 37 |
| half repeat | | |
| (G) | | |

HBG-A11 protein sequence:
(SEQ ID NO: 38)
MDYKDHDGDYKDHDIDYKDDDDKMAPKKKRKVGIHRGVPMVDLRTL
GYSQQQQEKIKPKVRSTVAQHHEALVGHGFTHAHIVALSQHPAALGTVA
VKYQDMIAALPEATHEAIVGVGKQWSGARALEALLTVAGELRGPPLQLD
TGQLLKIAKRGGVTAVEAVHAWRNALTGAPLNLTPDQVVAIASNIGGKQ
ALETVQRLLPVLCQDHGLTPDQVVAIASNGGGKQALETVQRLLPVLCQD
HGLTPDQVVAIASHDGGKQALETVQRLLPVLCQDHGLTPDQVVAIASHD
GGKQALETVQRLLPVLCQDHGLTPDQVVAIASNGGGKQALETVQRLLPV
LCQDHGLTPDQVVAIASHDGGKQALETVQRLLPVLCQDHGLTPDQVVAI
ASNGGGKQALETVQRLLPVLCQDHGLTPDQVVAIASNGGGKQALETVQR
LLPVLCQDHGLTPDQVVAIASNHGGKQALETVQRLLPVLCQDHGLTPDQ
VVAIASNHGGKQALETVQRLLPVLCQDHGLTPDQVVAIASNHGGKQALE
TVQRLLPVLCQDHGLTPDQVVAIASNHGGKQALETVQRLLPVLCQDHGL
TPDQVVAIASNHGGKQALETVQRLLPVLCQDHGLTPDQVVAIASHDGGK
QALETVQRLLPVLCQDHGLTPDQVVAIASHDGGKQALETVQRLLPVLCQ
DHGLTPDQVVAIASHDGGKQALETVQRLLPVLCQDHGLTPDQVVAIASH
DGGRPALESIVAQLSRPDPALAALTNDHLVALACLGGRPALDAVKKGLP
HAPALIKRTNRRIPERTSHRVAGS

### Example 1: Tale for Specific Regulation of a Target Gene

Transcription factors regulate gene expression. Gene expression can be artificially perturbed by expressing dominant negative forms of transcription factors that lack activation/repression domains. However, naturally occurring TFs bind to thousands of gene in the genome, making dominant negative forms of TFs unsuitable for targeting a specific gene. Provided herein are DNA binding proteins that not only displace an endogenous TF but are specific for a target gene. The DNA binding protein replaces endogenous TF from only a target gene since it binds to a sequence present only in the target gene. TALE proteins that specifically bind to a sequence in a target gene were designed and tested.

β-hemoglobinopathies, such as, sickle cell disease, β-thalassemia, are caused by mutations in the adult β-globin gene. During development, globin genes undergo switching: adult β-globin is expressed and the fetal γ-globin promoter is directly bound and silenced by the transcription factors ZBTB7A and BCL11A (Bauer, D. et al., Blood 2012). Mutations in the fetal γ-globin promoter result in incomplete silencing of the fetal γ-globin promoter (Hereditary Persistence of Fetal Hemoglobin; HPFH). HPFH patients who inherit sickle cell gene (Hb S) show symptomatic amelioration of sickle cell disease. Accordingly, reactivation of fetal globin expression to increase level of functional Hb is avidly being sought as a therapeutic avenue.

Mutations in HPFH patients cluster in TF binding sites. TALE proteins that bind to TF binding sites were designed to test the hypothesis that displacing TFs bound to γ-globin proximal promoter may reactivate fetal γ-globin expression.

TALEs A11 that binds to a nucleotide sequence spanning the binding site for the TF ZBTB7A was designed. A11 TALEs induced increase in relative expression of HBG as compared to total hemoglobin (adult hemoglobin (HBB)+HBG). Data not shown.

### Example 2: Tales Encoded Using Diversified Nucleic Acid Sequences

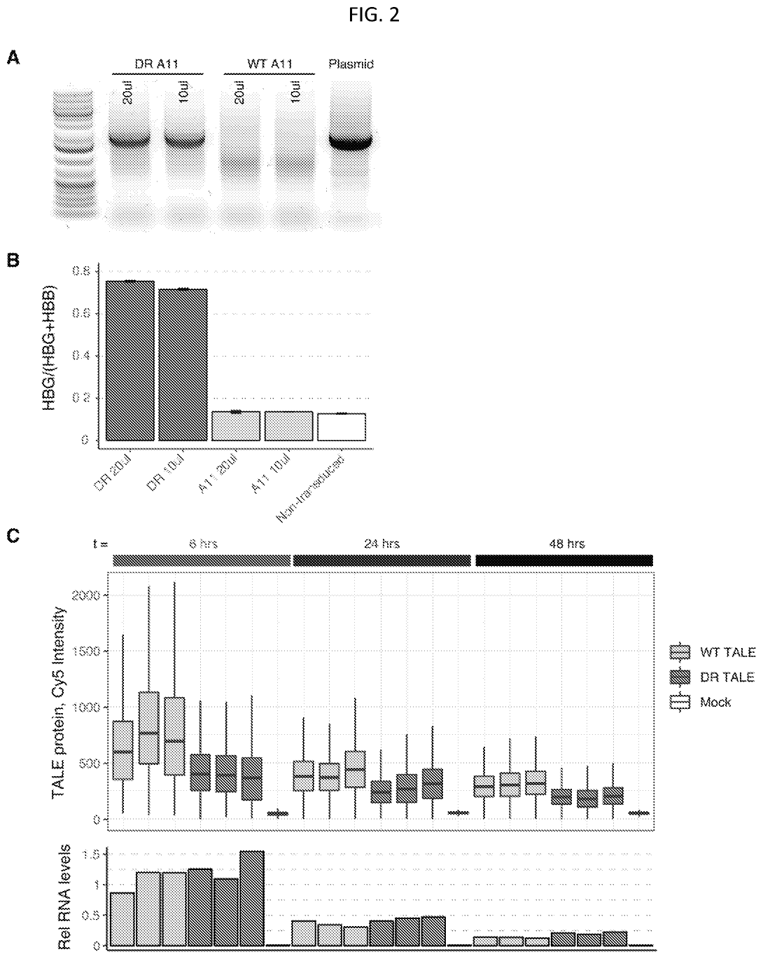

Nucleic acid sequences encoding the repeat units of the TALEs were diversified to reduce recombination and elimination of the encoding sequence in vivo. For example, when lentivirus is used to deliver the TALE encoding nucleic acid into cells, some of the nucleic acid sequence can be recombined resulting in decreased expression of the encoded TALE. The diversified repeat (DR) nucleic acid encoding a TALE can be delivered by lentivirus and result in fetal globin upregulation (FIG. 2, panel B) and sustained expression (panel C). As compared to the undiversified sequence (“WTA11”), the diversified sequence (“DRA11”) provides for stable lentiviral vector that retains the TALE encoding sequence (FIG. 2, panel A). The higher level of non-recombined lentiviral vector results in higher expression level of the TALE protein as evident by the significant increase in HBG expression when using lentivirus to deliver the diversified nucleic acid sequence (“DR”) as compared to when using lentivirus to deliver the undiversified nucleic acid sequence (“A11”).

Sequence of DRA11 nucleic acid encoding HBG-A11
TALE:
(SEQ ID NO: 39)
CTTACGCCTGACCAGGTGGTAGCGATAGCCAGCAACATTGGGGGGAA
ACAAGCACTCGAAACTGTTCAGCGACTGCTCCCTGTTTTGTGCCAAGAC
CACGGATTGACTCCCGACCAAGTGGTTGCTATCGCCAGCAACGGAGGGG
GTAAGCAGGCACTGGAAACCGTTCAAAGGCTGTTGCCAGTTCTCTGCCA
GGATCATGGATTGACACCGGACCAAGTTGTAGCAATCGCCAGCCACGAT
GGCGGCAAGCAAGCACTGGAAACAGTGCAAAGATTGCTGCCCGTTCTTT
GCCAGGACCACGGTCTTACACCAGACCAAGTAGTGGCAATCGCGAGTCA
CGATGGTGGTAAGCAGGCTCTTGAAACTGTACAGCGGCTGTTGCCTGTG
TTGTGTCAAGATCATGGACTTACGCCAGATCAGGTGGTGGCCATCGCGT
CAAATGGTGGGGGAAAGCAAGCGCTCGAAACGGTTCAGAGGCTCCTCCC
GGTTCTGTGTCAGGATCATGGGCTGACACCGGATCAAGTGGTAGCAATT
GCCTCACACGACGGGGGGAAACAGGCGCTTGAAACGGTCCAACGGCTGC
TCCCTGTGCTTTGCCAAGATCACGGCCTGACCCCGGATCAAGTAGTGGC
TATTGCTAGCAATGGTGGGGGTAAGCAAGCTCTGGAGACtGTGCAGAGA
CTTCTCCCTGTTCTTTGTCAAGATCACGGACTTACACCGGACCAGGTAG
TTGCAATCGCGAGCAACGGAGGCGGGAAACAAGCTCTCGAAACTGTACA
AAGGCTTCTCCCAGTACTTTGTCAGGATCACGGCcttACGCCCGACCAG
GTTGTAGCCATTGCCAGCAACCACGGGGGGAAGCAAGCCCTCGAAACCG
TTCAGAGGCTTCTGCCTGTGCTTTGTCAGGACCACGGACTCACCCCTGA
TCAAGTCGTGGCTATCGCCAGTAACCACGGGGGAAAACAAGCCCTTGAA
ACGGTGCAAAGACTTCTTCCGGTGCTGTGTCAGGACCATGGGCTTACGC
CAGACCAGGTGGTGGCGATTGCCAGTAATCATGGCGGTAAGCAGGCGCT
CGAAACTGTGCAGCGGCTGCTGCCGGTTCTTTGCCAAGACCATGGACTT
ACCCCCGATCAAGTGGTTGCCATCGCGAGTAATCACGGCGGCAAACAGG
CGCTGGAAACGGTACAACGGCTGTTGCCGGTCCTTTGCCAGGATCACGG
GcttACGCCTGATCAAGTTGTTGCGATCGCCAGCAATCACGGGGGCAAG
CAAGCTCTTGAAACGGTTCAAAGACTGCTCCCGGTTTTGTGTCAAGACC
ATGGTTTGACCCCTGACCAAGTTGTTGCAATTGCCAGCCACGACGGTGG
TAAACAGGCTCTCGAAACAGTCCAAAGGCTTTTGCCGGTACTCTGTCAA
GACCACGGCCTTACTCCGGACCAGGTGGTTGCCATTGCGAGTCACGACG
GGGGCAAACAGGCACTCGAAACGGTCCAGAGACTTTTGCCTGTGCTCTG
CCAAGATCATGGTCTGACTCCTGACCAAGTGGTGGCAATCGCCTCACAC
GATGGTGGGAAGCAGGCCCTCGAAACGGTACAGCGACTGTTGCCCGTAT
TGTGCCAGGACCATGGCCTGACGCCGGACCAGGTTGTGGCCATAGCTAG
CCACGATGGAGGA

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims. It is also to be understood that the terminology used herein is for the purpose of describing particular aspects only, and is not intended to be limiting, since the scope of the present invention will be limited only by the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

What is claimed is:

1. A computer-implemented method for diversifying a nucleic acid sequence comprising a plurality of contiguous stretches of identical sequences, wherein the nucleic acid sequence encodes a protein comprising a plurality of repeating amino acid sequences, the method comprising:
a) identifying in the nucleic acid sequence a 1^{st} contiguous stretch of nucleotides (nts) that is identical to a 2^{nd} contiguous stretch of nts and is longer than 14 nts;
b) replacing a codon in the 2^{nd} contiguous stretch with a different codon encoding the same amino acid;
c) determining whether the replaced codon introduces a restriction enzyme (RE) site, and
retaining the replaced codon if a RE site is not introduced; or
reverting to the original codon if a RE site is introduced; and
d) repeating steps a)-c) until a diversified nucleic acid sequence is generated which diversified nucleic acid sequence does not contain a pair of identical contiguous stretches of nts that are longer than 14 nts.

2. The computer-implemented method of claim 1, wherein step d) is performed at least 50 times, at least 100 times, or at least 500 times.

3. The computer-implemented method of claim 1 or 2, wherein step d) is performed up to 2000 times or up to 1000 times.

4. The computer-implemented method of any one of claims 1-3, wherein steps a)-d) are performed at least 5 times, at least 10 times, at least 100 times, or at least 500 times on the initial nucleic acid sequence.

5. The computer-implemented method of any one of claims 1-4, wherein steps a)-d) are performed up to 100 times or up to 200 times on the initial nucleic acid sequence.

6. The computer-implemented method of claim 4 or 5, where the steps a)-d) are performed simultaneously.

7. The computer-implemented method of any one of claims 1-6, wherein the method is performed in less than 5 hours, less than 1 hours, less than 30 minutes, less than 10 minutes, or less than 1 minute.

8. The computer-implemented method of any one of claims 1-7, wherein the method generates a plurality of different diversified nucleic acid sequences from the initial nucleic acid sequence.

9. The computer-implemented method of claim 8, the method further comprising selecting from the plurality of different diversified nucleic acid sequences a diversified nucleic acid sequence that comprises the shortest-longest-pair of identical contiguous stretches of nts.

10. The computer-implemented method of claim 8, the method further comprising selecting from the plurality of different diversified nucleic acid sequences a diversified nucleic acid sequence that includes the largest minimum-percent-divergence.

11. The computer-implemented method of claim 8, the method further comprising selecting from the plurality of different diversified nucleic acid sequences a diversified nucleic acid sequence that comprises a ratio of codons that is most similar to the ratio of codons in a cell in which the protein encoded by the diversified nucleic acid is to be expressed.

12. The computer-implemented method of 8, the method further comprising ranking the plurality of different diversified nucleic acid sequences from highest to lowest based on length of an identical pair of stretches of nts, wherein the diversified nucleic acid sequence that includes shortest-longest identical pair of contiguous stretch of nucleotides is ranked the highest and the diversified nucleic acid sequence that includes the longest-longest identical pair of contiguous stretch of nucleotides is ranked the lowest.

13. The computer-implemented method of claim 12, the method further comprising ranking the top half of the ranked plurality of different diversified nucleic acid sequences from largest to smallest minimum-percent-divergence, wherein the diversified nucleic acid sequence that has the largest-minimum-percent-divergence between the nucleic acid segments is ranked the highest and the diversified nucleic acid sequence that has the smallest minimum-percent-divergence is ranked the lowest.

14. The computer-implemented method of claim 13, the method further comprising ranking the top half of the plurality of different diversified nucleic acid sequences ranked according to minimum-percent-divergence, wherein the further ranking is based on ratio of codons and a diversified nucleic acid sequence comprising ratio of codons most similar to the ratio of codons in a cell in which the encoded protein is to be expressed is ranked higher than a diversified nucleic acid sequence comprising ratio of codons less similar to the ratio of codons in the cell in which the encoded protein is to be expressed.

15. The computer-implemented method of any one of claims 1-14, wherein the protein comprises a DNA binding domain comprising the repeating amino acid sequences.

16. The computer-implemented method of claim 15, wherein the repeating amino acid sequences are transcription activator-like effector (TALE) repeat units (RUs).

17. The computer-implemented method of claim 16, wherein the plurality of contiguous stretches of identical sequences each encode a part of or the entirety of a RU.

18. The computer-implemented method of claim 17, wherein the nucleic acid sequence comprises a plurality of 6 nts that encode a repeat variable diresidue (RVD) present in each RU, wherein the 6 nts are marked as non-replaceable and are not replaced.

19. The computer-implemented method of claim 18, wherein the plurality of 6 nts are identical in sequence.

20. The computer-implemented method of any one of claims 16-19, wherein each of the nucleic acid sequence comprises a plurality of nucleic acid segments up to 102 nts long, wherein each segment encodes a RU.

21. The computer-implemented method of any one of claims 16-20, wherein the nucleic acid sequence comprises up to 30 nucleic acid segments.

22. The computer-implemented method of any one of claims 16-20, wherein the plurality of nucleic acid segments comprise up to 22 nucleic acid segments.

23. The computer-implemented method of any one of claims 16-20, wherein the plurality of nucleic acid segments comprise up to 19 nucleic acid segments.

24. The computer-implemented method of any one of claims 16-23, wherein the plurality of nucleic acid segments are identical in length.

25. The computer-implemented method of claim 16-24, wherein the plurality of nucleic acid segments are arranged from the 5′ to the 3′ end of the nucleic acid sequence and wherein the last nucleic acid segment encodes a half RU and comprises a length that is approximately half of the length of the other nucleic acid segments.

26. The computer-implemented method of any one of claims 1-25, wherein the RE site is a site for EcoRI, BamHI, BsaI, BsmBI, AflII, XbaI, KpnI, ApaI, NheI, HindIII, NdeI, EcoRV, EagI, SspI, BspHI, or AleI.

27. The computer-implemented method of any one of claims 1-26, further comprising synthesizing a nucleic acid comprising the diversified nucleic acid sequence.

28. The computer-implemented method of any one of claims 1-26, further comprising synthesizing a plurality of nucleic acids comprising portions of the diversified nucleic acid sequence.

29. A method for generating a nucleic acid encoding a protein comprising a plurality of repeating amino acid sequences, wherein the nucleic acid comprises a sequence where no contiguous stretches of nucleotides that are identical to another contiguous stretch of nts and are longer than 14 nts are present, the method comprising:
inputting an initial nucleic acid sequence encoding the protein and comprising a plurality of contiguous stretches of identical sequences that are longer than 14 nts into a computer, wherein the computer performs the method of any one of claims 1-26 and outputs the sequence for the nucleic acid; and
generating the nucleic acid comprising the outputted sequence.


SEQUENCE LISTING (2 sequences)

SEQ ID NO: 1 - protein, 6 aa, synthetic construct
HHHHHH

SEQ ID NO: 2 - protein, 19 aa, synthetic construct
  MOD_RES 3: note = PHOSPHORYLATION
  MOD_RES 13: note = PHOSPHORYLATION
  UNSURE 9..10: note = PEG8 linker
LNYAQLWHALTIYATIRRF